SEQUENCE LISTING (8 sequences)

SEQ ID NO: 1 - protein, 6 aa, synthetic construct
  REGION 1..6: note = self-assembling peptide
VGVAPG

SEQ ID NO: 2 - protein, 6 aa, synthetic construct
  REGION 1..6: note = self-assembling peptide
IGVAPG

SEQ ID NO: 3 - protein, 5 aa, synthetic construct
  REGION 1..5: note = self-assembling peptide
KTTKS

SEQ ID NO: 4 - protein, 16 aa, synthetic construct
  REGION 1..16: note = self-assembling peptide
RADARADARADARADA

SEQ ID NO: 5 - protein, 13 aa, synthetic construct
  REGION 1..13: note = self-assembling peptide
IEIKIEIKIEIKI

SEQ ID NO: 6 - protein, 12 aa, synthetic construct
  REGION 1..12: note = self-assembling peptide
KLDLKLDLKLDL

SEQ ID NO: 7 - protein, 7 aa, synthetic construct
  REGION 1..7: note = self-assembling peptide
AAAAAAK

SEQ ID NO: 8 - protein, 4 aa, synthetic construct
  REGION 1..4: note = self-assembling peptide
FSRY

(19) United States
(12) Patent Application Publication
(10) Pub. No.: US 2024/0050329 A1
(43) Pub. Date: Feb. 15, 2024

(54) METHODS FOR COSMETIC SKIN REMODELING

(71) Applicant: ELC Management LLC, Melville, NY (US)

(72) Inventors: Kurt Matthew Schilling, Rochester, NY (US); Sierra BECK, Bayville, NY (US); Jaime Ann EMMETSBERGER, Manorville, NY (US); Melanie Ann HUTNICK, Melville, NY (US); Matthew Tyler IOVINO, Tyngsborough, MA (US); Jaimie Sabrina JERMOE, Setauket, NY (US); Nevena Mladen KARAMAN-JURUKOVSKA, Stony Brook, NY (US); Thomas MAMMONE, Farmingdale, NY (US); Fetemeh MOHAMMADI, Hauppauge, NY (US); Tsung-Wei Robert MOU, Stony Brook, NY (US); Elham TAVASOLI, Plainview, NY (US)

(21) Appl. No.: 18/223,174

(22) Filed: Jul. 18, 2023

Related U.S. Application Data
(63) Continuation of application No. 17/066,476, filed on Oct. 8, 2020.
(60) Provisional application No. 62/914,201, filed on Oct. 11, 2019.

(51) Int. Cl. A61K 8/02; A61Q 19/08

(52) U.S. Cl. CPC A61K 8/0283; A61Q 19/08; A61K 2800/10; A61K 2800/95; A61K 2800/805; A61K 2800/884; A61K 2800/74

(57) ABSTRACT

The methods described herein provide self-assembling macromolecular networks within the human skin. The methods provide instant and accumulation-based physical properties within the skin, causing cosmetic benefits including, bulking/plumping, filling, strengthening, smoothing, firming, and lifting, as well as enhancing the optical properties, radiance and other cosmetic appearance characteristics of the skin. The disclosure also provides a method of re-structuring the skin without cellular biogenesis by effecting macrostructures within the skin. The method comprising effectuating self-assembly within the skin layers by topically applying a composition comprising at least one self-assembly material to form functional macrostructures within the skin and effectuating self-assembly of the at least one self-assembly material in vivo while substantially preventing self-assembly prior to the topical application or treatment.

### APPLICATION PRIORITY INFORMATION

This application is a Divisional Application of copending U.S. patent application Ser. No. 17/066,476 filed on Oct. 8, 2020, wherein U.S. patent application Ser. No. 17/066,476 claims priority to U.S. Provisional Patent Application No. 62/914,201 filed Oct. 11, 2019, the contents are incorporated herein by reference in its entirety.

### FIELD OF THE INVENTION

The disclosure relates to the field of cosmetic or dermatological compositions. More particularly, the disclosure relates to the field of cosmetic compositions comprising at least one self-assembling material that self-assemble into macrostructures within the layers of the skin.

### REFERENCE TO SEQUENCE LISTING SUBMITTED ELECTRONICALLY

The instant application contains a Sequence Listing which was submitted electronically in XML format on Jul. 18, 2023 and is hereby incorporated by reference in its entirety. The XML file, created on Jul. 18, 2023, is named 18-21D1-Seq_Listing.xml and is 11 KB in size. This sequence listing submitted via EFS-Web is part of the specification and is herein incorporated by reference in its entirety.

### BACKGROUND OF THE INVENTION

Self-assembly is an elegant and expedient “bottom-up” approach towards designing ordered, three-dimensional and biocompatible nanobiomaterials. Reproducible macromolecular nanostructures can be obtained due to the highly specific interactions between the building blocks. These intermolecular associations organize the supramolecular architecture and are mainly non-covalent electrostatic interactions, hydrogen bonds, van der Waals forces, etc. Supramolecular chemistry or biology gathers a vast body of two-or three-dimensional (2D or 3D) complex structures and entities formed by association of chemical or biological species. These associations are governed by the principles of molecular complementarity or molecular recognition and self-assembly. The knowledge of the rules of intermolecular association can be used to design polymolecular assemblies in the form of membranes, films, layers, micelles, tubules, or gels for a variety of biomedical or technological applications. In biological systems these self-assembly molecules are generally lipids, peptides, proteins, sugars, and nucleic acids.

Peptides are versatile building blocks for fabricating supramolecular architectures. Their ability to adopt specific secondary structures, as prescribed by amino acid sequence, provides a unique platform for the design of self-assembling biomaterials with hierarchical 3D macromolecular architectures, nanoscale to macroscale features and tunable physical properties. Peptides are for instance able to assemble into nanotubes (U.S. Pat. No. 7,179,784) or into supramolecular hydrogels consisting of 3D scaffolds with a large amount of around 98-99% immobilized water or aqueous solution. The peptide-based biomaterials are powerful tools for potential applications in biotechnology, medicine and other technical applications. Depending on the individual properties these peptide-based hydrogels are thought to serve in the development of new materials for tissue engineering, regenerative medicine, as drug and vaccine delivery vehicles or as peptide chips for pharmaceutical research and diagnosis. There is also a strong interest to use peptide-based self-assembled biomaterial such as gels for the development of molecular electronic devices.

A variety of “smart peptide hydrogels” have been generated that react to external manipulations such as temperature, pH, mechanical influences or other stimuli with a dynamic behavior of swelling, shrinking or decomposing. Nevertheless, these biomaterials are still not “advanced” enough to mimic the biological variability of natural tissues as for example the extracellular matrix (ECM) or cartilage tissue or others. The challenge for a meaningful use of peptide hydrogels is to mimic the replacing natural tissues not only as “space filler” or mechanical scaffold, but to understand and cope with the biochemical signals and physiological requirements that keep the containing cells in the right place and under “in vivo” conditions.

Much effort has been undertaken to understand and control the relationship between peptide sequence and structure for a rational design of suitable hydrogels. In general hydrogels contain macroscopic structures such as fibers that entangle and form meshes. Most of the peptide-based hydrogels utilize 0-pleated sheets which assemble to fibers as building blocks. It is also possible to obtain self-assembled hydrogels from α-helical peptides besides (3-sheet structure-based materials.

Nevertheless, the currently known peptide hydrogels are in most of the cases associated with low rigidity, sometimes unfavorable physiological properties and/or complexity and the requirement of substantial processing thereof which leads to high production costs. There is therefore a widely recognized need for peptide hydrogels that are easily formed, non-toxic and have a sufficiently high rigidity for standard applications. The hydrogels should also be suitable for the delivery of bioactive moieties (such as nucleic acids, small molecule therapeutics, cosmetic and anti-microbial agents) and/or for use as biomimetic scaffolds that support the in vivo and in vitro growth of cells and facilitate the regeneration of native tissue and/or for use in 2D and/or 3D biofabrication.

Self-assembly is a powerful design template to create complex molecular assemblies from a limited number of building blocks. There has been little or no reports of utilizing self-assembling networks for topical application to skin and scalp with the intent to create systems that form supramolecular structures at designated spaces in or on skin tissue. These applications have complex requirements for any self-assembling system, including personal care, cosmetic methods and formulations, its compatibility and stability, application to the outside of the target tissue, penetration to the right site or layer in the skin or scalp, the inherent physio-chemical properties inside the skin layer, and the need to control where the self-assembly occurs between package surface, formulation, on skin surface or inside the skin.

### SUMMARY OF THE INVENTION

The methods described herein provide self-assembling macromolecular networks within the human skin. The methods provide accumulation-based physical properties within the skin, causing cosmetic benefits including, bulking/plumping, filling, strengthening, smoothing, firming, and lifting, as well as enhancing the optical properties, radiance and other cosmetic appearance characteristics of the skin. The disclosure provides a method of re-structuring the skin without cellular biogenesis by affecting macrostructures within the skin. The method comprises effectuating self-assembly within the skin layers by applying a cosmetic composition comprising at least one self-assembly material, wherein the self-assembly material forms functional macrostructures within the skin. The method further comprises effectuating the self-assembly of the at least one self-assembly material in vivo while substantially preventing the self-assembly of the at least one self-assembly material in the composition prior to the topical application or treatment. The self-assembly material comprises at least one oligomer. The self-assembly material further comprises more than one oligomer that can assemble with each other in the skin. The method provides at least one cosmetic skincare, hair care or make up benefit. The cosmetic benefit includes improving wrinkles, fine lines, pores, sagginess, anti-aging, bulking/plumping, filling, barrier strengthening, smoothing, firming, optical properties of skin, lifting, radiance or glow. The functional macrostructures are 3D and the oligomer forms functional macrostructures within the skin following a trigger. The trigger is temperature, ionic strength, concentration, pH, solvent or combinations thereof or an application of a second composition prior to or following the topical application or treatment. Specifically, the trigger includes a pH in a range of about 3 to 9, a change in the pH value of 0.001 to 5 in pH units, a gradient of temperature, pH, salt or ions that exists in the skin layers, a change in the salt concentration, or a change in the concentration of the self-assembly material. Specifically, the pH trigger is a change in the pH value from the non-physiological pH to the physiological pH value within the skin layers. The self-assembly is effectuated after the cosmetic composition is applied topically within 6 hours following the topical application.

### BRIEF DESCRIPTION OF THE DRAWINGS

FIG. 1 shows the results of elastin fiber measurements obtained from histological images as well as the corresponding analyses. FIG. 1A shows the Elastin van Gieson staining showed temperature driven co-assembling of the elastin-like peptides (ELPs) to existing elastin fibers compared to dermises. FIG. 1B shows the quantification of elastin fiber density in decellularize dermises.

FIG. 2 shows the representative images of ex vivo skin treated topically with various concentrations of the ELPs, Pal-VGVAPG and Ace-IGVAPG.

FIG. 3 shows the results of click histology.

FIG. 4 shows the effect of salinity of peptide self-assembly.

FIG. 5 shows the combined representative results of Pal-VGVAPG for pH and salinity

FIG. 6 demonstrates a cosmetic benefit of increased skin elasticity via self-assembly and co-assembly of peptides, ex vivo skin.

### DETAILED DESCRIPTION OF THE INVENTION

All percentages and ratios used herein are by weight of the total composition and all measurements made are at 25° C., unless otherwise designated. All numeric ranges are inclusive of narrower ranges; delineated upper and lower range limits are interchangeable to create further ranges not explicitly delineated.

The compositions can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The terms “apply” or “application” as used in reference to a composition, means to contact or spread the compositions onto a substrate such as the human skin surface or epidermis.

The term “dermatologically or cosmetically acceptable” as used herein means that the compositions or components described are suitable for use in contact with human skin tissue without undue toxicity, incompatibility, instability, allergic response, and the like.

The term “facial skin surface” as used herein refers to one or more of forehead, periorbital, cheek, perioral, chin, and nose skin surfaces. While facial skin surfaces are of concern and are exemplified herein, other skin surfaces may be treated with the compositions, for example, surfaces typically not covered by clothing such as facial skin surfaces, hand and arm skin surfaces, foot and leg skin surfaces, and neck and chest skin surfaces (e.g., décolletage).

The term “keratinous tissue,” as used herein, refers to keratin-containing layers disposed as the outermost protective covering of mammals, which includes, but is not limited to, skin, scalp, mucosa, lips, hair, toenails, fingernails, cuticles, hooves, etc.

The terms “topical application”, “topically”, and “topical”, as used herein, mean to apply (e.g., spread, spray) the compositions onto the surface of the keratinous tissue.

As used herein, “effective amount” means an amount of a compound or composition sufficient to significantly induce a positive keratinous tissue benefit, including independently or in combination with other benefits disclosed herein. This means that the content and/or concentration of agent in the formulation is sufficient that when the formulation is applied with normal frequency and in a normal amount, the formulation can result in the treatment of one or more undesired keratinous tissue conditions (e.g. skin wrinkles). For instance, the amount can be an amount sufficient to inhibit or enhance some biochemical function occurring within the keratinous tissue. This amount of the skin care agent may vary depending upon the type of product, the type of keratinous tissue condition to be addressed, and the like.

The term “self-assembly” as used herein, refers to materials, such as, without limitation, molecules, biomolecules, DNA, polysaccharides, polymers, colloids, or macroscopic particles that organize and effectuate into ordered and/or functional and well-defined 2D or 3D macrostructures or patterns as a consequence of specific, local interactions. Such 2D or 3D macrostructures or assemblies may directly impart benefits including, without limiting, physical properties to the tissue, skin layers such as volume, turgor, elasticity, light scattering, among others. While external direction is not required to physically order the materials, external conditions such as temperature, pressure, ionic strength, salt strength, pH, and other factors may influence whether self-assembly of certain materials may occur. In specific instances, for example, the self-assembly of molecules is defined as the ability of specific molecules such as, without limitation, oligomers of macromolecules to spontaneously bind non-covalently to themselves under appropriate condition and form large ordered well-defined 3D functional macrostructures within the skin layers or on or within the bodily tissues. The macrostructures may range from nanoscale or millimeter scale.

The term “co-assembly” as used herein, refers to molecules, biomolecules or materials, such as, without limitation, molecules, polymers, DNA, polysaccharides, colloids, or macroscopic particles that organize into ordered and/or functional and well-defined 2D or 3D macrostructures structures or patterns as a consequence of specific, local interactions among the components themselves. In specific instances, specific molecules such as oligomers of macromolecules spontaneously bind non-covalently to other complementary oligomers under appropriate condition and form binary system with larger 3D structure.

Further, the term “self-assembly” is meant to include the term “co-assembly”, as it relates to the formation of such self-directed ordered systems.

The term “self-assembly peptides” as used herein refers to a peptide or a combination of peptides formed from a chain of at least two amino acids (α-amino acid residues) linked by covalent bonds, such as peptide bonds, wherein the peptides self-assemble to form matrices upon contact with bodily tissues within the skin, on or within the skin tissues. Self-assembling peptides may be branched, in which case they will comprise at least two amino acid chains linked by a non-peptide bond. Further, self-assembling peptides can vary in length so long as they can self-assemble in vivo. While the amino acid sequences of the self-assembling peptides can vary, in certain instances, sequences may include those that provide an amphiphilic nature to the self-assembling peptides, for example, the self-assembling peptides can include, not necessarily, approximately equal numbers of hydrophobic and hydrophilic amino acids.

The term “macroscopic” refers to dimensions that are large enough to be visible under magnification of 10-fold or more. The macrostructures can be two dimensional or three dimensional.

The term “skin active agent” as used herein, refers to an active ingredient which provides a cosmetic and/or dermatological effect to the area of application on the skin.

The terms “stable” and “stability” as used herein mean a composition which is substantially unaltered in chemical state, physical homogeneity and/or color when the composition is at a temperature of from about 4° C. to about 50° C., with and/or without humidity conditions.

The term “subdermal” as used herein, means within or penetrating the epidermis.

The term “transdermal” as used herein, means the application of a material or composition through the surface of the skin.

Human skin is divided into three layers, the epidermis; the dermis, and the subcutaneous tissue or bottom layer. The epidermis includes an outer layer and an inner layer. At the base of the epidermis, cells called melanocytes (about 5% of the epidermal cells) exist. The dermis or the middle layer contains collagen and other materials vital to the skin's strength, its ability to repair itself. The subcutaneous layer or the bottom layer serves as insulation to the body.

Methods and cosmeceutical and dermatological preparations or compositions that include materials that self-assemble within the skin subsequent to topical administration are disclosed herein. The compositions comprise self-assembling molecules including, but not limited to, lipids, peptides, polysaccharides, nucleic acids, surfactants and its derivatives and other molecules including derivatives, individually or in combination to form a highly ordered structure or structures within the skin tissue or on the skin tissue present within the skin layers through non-invasive topical application. Such topical administration, thereby, achieves skin effects and cosmetic benefits such as, without limitation, lifting, firming, and plumping etc.

### Self-Assembly

Self-assembly is an arrangement or organization of molecules, under certain conditions into structurally well-defined arrangements. Such organization may occur spontaneously or may occur subsequent to the conditions that favor such self-assembly. Self-assembly of molecules is caused by non-covalent interactions and physical bonds. The non-covalent interactions include hydrogen bonds, ionic bonds, and van der Waals forces and the like, necessary to assemble the molecules into well-defined organized structures with complementarity and structural compatibility. The self-assembly materials are molecules including, but not limited to, oligomers of peptides, DNAs, polysaccharides, sugars, polymers and carbohydrates that can self-assembly into organized structures within the skin upon a trigger.

Self-assembly is substantially prevented from occurring in the composition prior to the application or treatment due to the conditions present in the formulation; however, the self-assembly occurs in vivo when an appropriate trigger is applied, or appropriate conditions or molecules exist and induce assembly in the skin layers. As a result of the self-arrangement of the molecules within the skin layers, various physical effects occur at the target layers of the skin, resulting in cosmetic benefits such as, skin lifting, firming, reduction of lines, affecting pores on the skin, wrinkles, thickening of epidermal layers, among others. Further, the methods and compositions also provide enhanced delivery of actives within the layers of the skin, thereby providing structural and physical enhancement to the skin through topical application. It is also an objective of the present disclosure to provide a topically applied method utilizing a novel, self-propagating mechanism that adds new structure to the skin, which changes the physical and mechanical properties within the layers of the skin and of the tissue without involving induction of new biogenesis by skin cells, including tissue or cellular biogenesis. Specifically, a physical phenomenon that enables re-structuring of the human skin by changing its physical and mechanical properties within the layers of the skin without engaging in cellular biogenesis and via topical application is described herein. The physical self-assembling phenomenon provides at least one skin benefit, which is perceived shortly after or following the treatment, preferably immediately or subsequent to the topical application. The physical self-assembling phenomenon also enables macroscopic re-structuring of the skin by changing the mechanical properties of skin at the desired location within the skin layers. The macroscopic structures or assemblies may be 2D or 3D. If 2D, the macroscopic structure comprises more than a single layer of molecules, for example, without limitation, at least two or more layers of molecules that aid in structural changes. The macrostructure assemblies may range from nanometers to millimeters in size. The macroscopic 2D assemblies or structures include, for example, nanoribbons or tapes, among others.

### Self-Assembly Materials

The self-assembly is the ability of oligomers of macromolecules (e.g., peptide, peptoid, protein, carbohydrate, oligonucleotide, DNA, RNA, lipid, and their derivatives, among others) to spontaneously bind non-covalently to themselves under appropriate condition and form ordered well-defined 2D or 3D structures. The self-assembling materials include molecules such as, without limitation, oligomers of peptides, DNAs, carbohydrates, lipids, synthetic hybrids, and/or combination of thereof that can self-assembly into organized structures within the skin upon a trigger.

When the self-assembling materials, for example, peptides, are administered topically on the skin, the materials migrate through the tissue and self-assemble within the layers of the skin in a controlled condition, i.e., a trigger of choice. The trigger of choice include a gradient condition that exists within skin layers; a change in a condition between the formulation and the skin layer, such as, for example a change in the value of temperature, pH, salinity, ionic strength or concentration of the peptides between the composition and the skin layers; a condition that exists within the skin layer, including in vivo temperature, pH, ions, salinity, ionic strength, enzyme concentration, peptide amounts; or molecules within the skin such as, for example, elastin, fibronectin, collagen, hyaluronic acid, among others. Similarly, mixtures of materials, such as, oligomer of peptides, multiple peptides, multiple classes of peptides or multiple oligomers can also be applied to achieve an effect. In some specific instances, multiple peptides of different classes or types, which otherwise do not bind with each other, when delivered within the skin layers begin to co-aggregate causing self-assembly. Thus, self-assembly is induced within the skin in vivo, caused by a trigger of choice, while the self-assembly is substantially prevented from occurring in the composition prior to the application or treatment.

Upon topical administration of the composition on the skin, the material migrates through the skin and upon delivery of the material within the skin layers, self-assembly is initiated by a trigger of choice as described herein and the self-assembly is induced. The trigger may be applied as a subsequent application of a different composition or by a gradient condition within the skin layers including, pH, temperature, salinity, ionic strength, enzyme concentration etc., or by a change in the value of such condition i.e., without limitation, change in the value of pH, ionic strength, salinity, solvent, enzyme concentration, light or temperature between the composition and the skin layers or by the molecules that exists within the skin layers. This physical phenomenon effectuates re-structuring of the skin by changing its mechanical properties at the desired location within the skin, without engaging in new cellular or tissue biogenesis. The methods and composition thereby, provides cosmetic or dermatological benefits such as, for example, reduction in lines and wrinkles, reduction in pores, increased skin firmness, elasticity and smoothness, increased plumping, radiance etc., by effectuating self-assembly within the skin and via effecting mechanical and physical properties of the skin.

A. Self-Assembling Peptides

A composition comprising oligomers, i.e., peptides capable of self-assembly within the skin is disclosed. The term “peptide,” as used herein includes “polypeptide,” “oligopeptide,” and “protein,” and refers to a string of at least two α-amino acid residues linked together by covalent bonds (e.g., peptide bonds). Such peptides can vary in length so long as they retain the ability to self-assemble to an extent useful for one or more of the purposes described herein. Peptides having as few as two α-amino acid residues or as many as approximately 200 residues may be suitable, and those recognized to self-assemble typically have a length within this range (e.g., 4-200, 8-36, 8-24, 8-16, 12-20, 6-64, 4-8, 5-10, 2-6, 1-8, 16-20, 2-42, 5-40, 10-40 amino acid residues, including, all ranges and subranges within).

“Peptide” may refer to an individual peptide or to a collection of peptides having the same or different sequences, any of which may include only naturally occurring α-amino acid residues, non-naturally occurring α-amino acid residues, or both. α-Amino acid analogs are also known in the art and may alternatively be employed. In particular, α-amino acid residues of the D-form may be used.

In addition, one or more of the amino acid residues in a self-assembling peptide can be altered or derivatized by the addition of a chemical entity such as an acyl group, an alkyl group, a carbohydrate group, a carbohydrate chain, a phosphate group, a farnesyl group, an isofarnesyl group, a fatty acid group, or a linker for conjugation or functionalization. Useful peptides can also be branched, in which case they will comprise at least two amino acid residues, each of which consists of at least three amino acid residues joined by peptide bonds. The two amino acid residues themselves may be linked, in some instance, not by a peptide bond. The amino acid residues in the self-assembling peptides can be naturally occurring or non-naturally occurring amino acid residues. Naturally occurring amino acids can include amino acid residues encoded by the standard genetic code as well as non-standard amino acids (e.g., amino acids having the D-configuration instead of the L-configuration), as well as those amino acids that can be formed by modifications of standard amino acids. Non-canonical or non-naturally occurring amino acids have not been found in nature but can be incorporated into a peptide chain.

Self-assembling peptides can be chemically synthesized, enriched or purified from natural or recombinantly-produced sources by methods well known in the art. For example, peptides can be synthesized using standard Fmoc chemistry and purified using high pressure liquid chromatography (HPLC). Tables 1, 2 and 3 include a non-limiting exemplary representation of self-assembling peptides. Table 1 indicates the sequence of exemplary peptides while Tables 2 and 3 show the chemical structures. Specifically, Table 2 refers to the respective chemical structures (SEQ.ID.NOs: 1-8 and sequence numbers #1-9) corresponding to Table 1. Table 3 refers to the backbone chemical structure of the respective sequences of Table 1.

TABLE 1

| | Exemplary Peptides | |
|---|---|---|
| # | Sequence | SEQ.ID.NOs |
| 1 | R¹-VGVAPG-R² | SEQ.ID.NO.: 1 |
| 2 | R¹-IGVAPG-R² | SEQ.ID.NO.: 2 |
| 3 | R¹-GH-R² | |
| 4 | R¹-KTTKS-R² | SEQ.ID.NO.: 3 |
| 5 | R¹-RADARADARADARADA-R² | SEQ.ID.NO.: 4 |
| 6 | R¹-IEIKIEIKIEIKI-R² | SEQ.ID.NO.: 5 |
| 7 | R¹-KLDLKLDLKLDL-R² | SEQ.ID.NO.: 6 |
| 8 | R¹-AAAAAAK-R² | SEQ.ID.NO.: 7 |
| 9 | R¹-FSRY-R² | SEQ.ID.NO.: 8 |

The Sequence Listing file in ST.26 format, identified as: 18-21D1_Seq_Listing.xml, file created Jul. 18, 2023, is hereby incorporated by reference, in its entirety.

TABLE 2
Peptide Structures

| Name | CHEMICAL STRUCTURES |
|---|---|
| Hexapeptide | 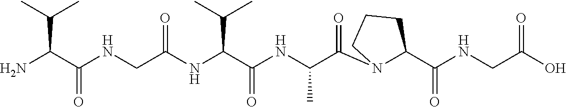 [SMILES: CC(C)[C@H](N)C(=O)NCC(=O)N[C@H](C(=O)N[C@@H](C)C(=O)N1CCC[C@H]1C(=O)NCC(=O)O)C(C)C] |
| Palmitoyl Hexapeptide | 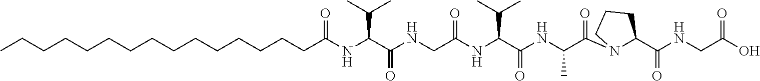 [SMILES: CCCCCCCCCCCCCCCC(=O)N[C@H](C(=O)NCC(=O)N[C@H](C(=O)N[C@@H](C)C(=O)N1CCC[C@H]1C(=O)NCC(=O)O)C(C)C)C(C)C] |
| Elastin Hexapeptide | 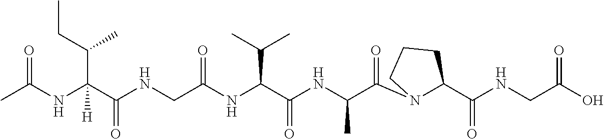 [SMILES: CC[C@H](C)[C@H](NC(C)=O)C(=O)NCC(=O)N[C@H](C(=O)N[C@H](C)C(=O)N1CCC[C@H]1C(=O)NCC(=O)O)C(C)C] |
| Palmitoyl Dipeptide-18 | 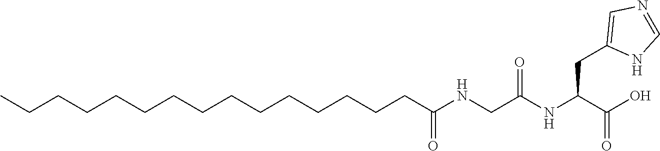 [SMILES: CCCCCCCCCCCCCCCC(=O)NCC(=O)N[C@@H](Cc1cnc[nH]1)C(=O)O] |
| Palmitoyl Pentapeptide-4 | 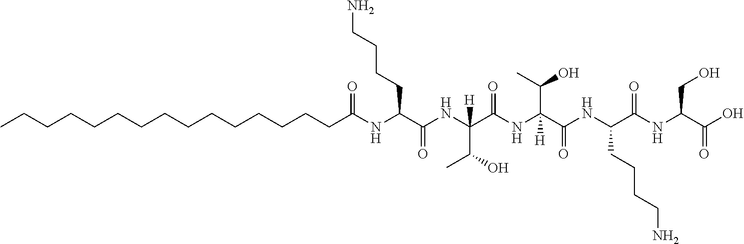 [SMILES: CCCCCCCCCCCCCCCC(=O)N[C@@H](CCCCN)C(=O)N[C@H](C(=O)N[C@H](C(=O)N[C@@H](CCCCN)C(=O)N[C@@H](CO)C(=O)O)[C@@H](C)O)[C@@H](C)O] |
| RADA-16 | 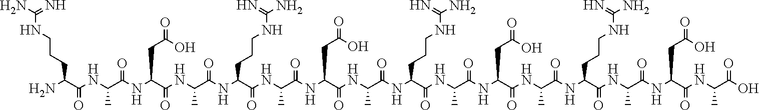 [SMILES: C[C@H](NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@@H](N)CCCNC(=N)N)C(=O)O] |
| IEIK-13 | 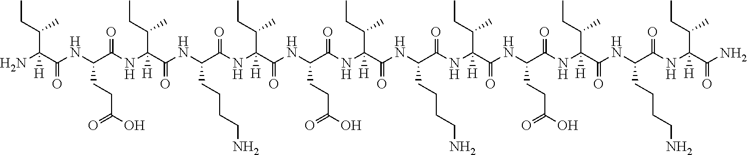 [SMILES: CC[C@H](C)[C@H](N)C(=O)N[C@@H](CCC(=O)O)C(=O)N[C@H](C(=O)N[C@@H](CCCCN)C(=O)N[C@H](C(=O)N[C@@H](CCC(=O)O)C(=O)N[C@H](C(=O)N[C@@H](CCCCN)C(=O)N[C@H](C(=O)N[C@@H](CCC(=O)O)C(=O)N[C@H](C(=O)N[C@@H](CCCCN)C(=O)N[C@H](C(N)=O)[C@@H](C)CC)[C@@H](C)CC)[C@@H](C)CC)[C@@H](C)CC)[C@@H](C)CC)[C@@H](C)CC] |
| KLD-12 | 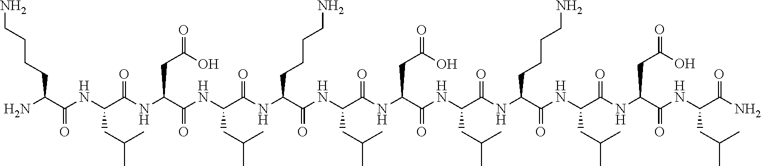 [SMILES: CC(C)C[C@H](NC(=O)[C@H](CC(=O)O)NC(=O)[C@H](CC(C)C)NC(=O)[C@H](CCCCN)NC(=O)[C@H](CC(C)C)NC(=O)[C@H](CC(=O)O)NC(=O)[C@H](CC(C)C)NC(=O)[C@H](CCCCN)NC(=O)[C@H](CC(C)C)NC(=O)[C@H](CC(=O)O)NC(=O)[C@H](CC(C)C)NC(=O)[C@@H](N)CCCCN)C(N)=O] |
| A6K | 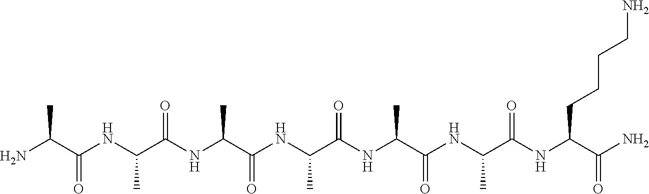 [SMILES: C[C@H](N)C(=O)N[C@@H](C)C(=O)N[C@@H](C)C(=O)N[C@@H](C)C(=O)N[C@@H](C)C(=O)N[C@@H](C)C(=O)N[C@@H](CCCCN)C(N)=O] |
| Palmitoyl Tetrapeptide | 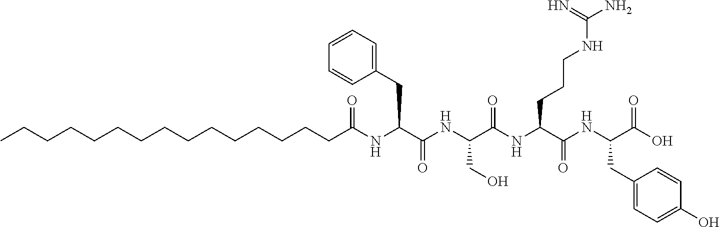 [SMILES: CCCCCCCCCCCCCCCC(=O)N[C@@H](Cc1ccccc1)C(=O)N[C@@H](CO)C(=O)N[C@@H](CCCNC(=N)N)C(=O)N[C@@H](Cc1ccc(O)cc1)C(=O)O] |

TABLE 3
Backbone Peptide Structures Corresponding to Table 1

| Backbone of: | CHEMICAL STRUCTURES |
|---|---|
| Hexapeptide | 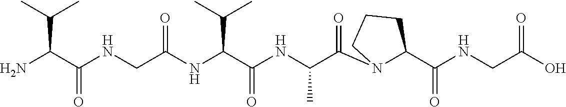 [SMILES: CC(C)[C@H](N)C(=O)NCC(=O)N[C@H](C(=O)N[C@@H](C)C(=O)N1CCC[C@H]1C(=O)NCC(=O)O)C(C)C] |
| Elastin hexapeptide | 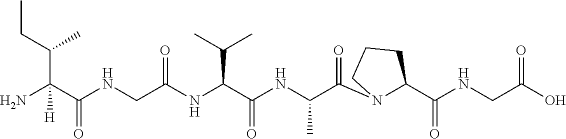 [SMILES: CC[C@H](C)[C@H](N)C(=O)NCC(=O)N[C@H](C(=O)N[C@@H](C)C(=O)N1CCC[C@H]1C(=O)NCC(=O)O)C(C)C] |
| Dipeptide | 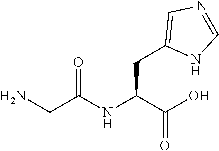 [SMILES: NCC(=O)N[C@@H](Cc1cnc[nH]1)C(=O)O] |
| Pentapeptide | 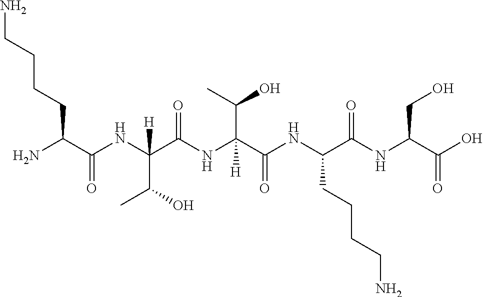 [SMILES: C[C@@H](O)[C@H](NC(=O)[C@@H](NC(=O)[C@@H](N)CCCCN)[C@@H](C)O)C(=O)N[C@@H](CCCCN)C(=O)N[C@@H](CO)C(=O)O] |
| RADA-16 | 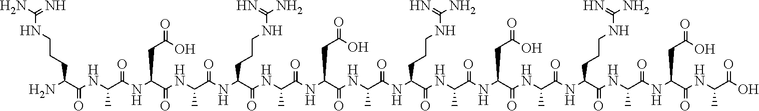 [SMILES: C[C@H](NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@H](CCCNC(=N)N)NC(=O)[C@H](C)NC(=O)[C@H](CC(=O)O)NC(=O)[C@H](C)NC(=O)[C@@H](N)CCCNC(=N)N)C(=O)O] |
| IEIK-13 | 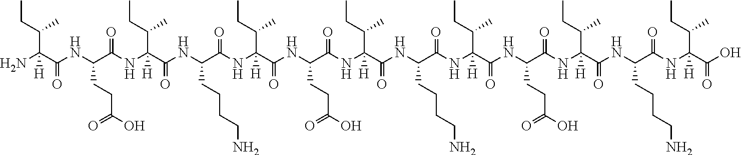 [SMILES: CC[C@H](C)[C@H](N)C(=O)N[C@@H](CCC(=O)O)C(=O)N[C@H](C(=O)N[C@@H](CCCCN)C(=O)N[C@H](C(=O)N[C@@H](CCC(=O)O)C(=O)N[C@H](C(=O)N[C@@H](CCCCN)C(=O)N[C@H](C(=O)N[C@@H](CCC(=O)O)C(=O)N[C@H](C(=O)N[C@@H](CCCCN)C(=O)N[C@H](C(=O)O)[C@@H](C)CC)[C@@H](C)CC)[C@@H](C)CC)[C@@H](C)CC)[C@@H](C)CC)[C@@H](C)CC] |
| KLD-12 | 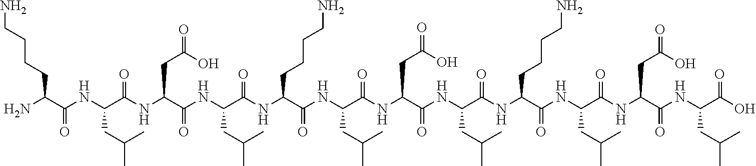 [SMILES: CC(C)C[C@H](NC(=O)[C@H](CC(=O)O)NC(=O)[C@H](CC(C)C)NC(=O)[C@H](CCCCN)NC(=O)[C@H](CC(C)C)NC(=O)[C@H](CC(=O)O)NC(=O)[C@H](CC(C)C)NC(=O)[C@H](CCCCN)NC(=O)[C@H](CC(C)C)NC(=O)[C@H](CC(=O)O)NC(=O)[C@H](CC(C)C)NC(=O)[C@@H](N)CCCCN)C(=O)O] |
| A6K | 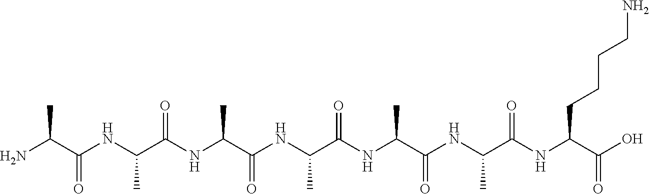 [SMILES: C[C@H](N)C(=O)N[C@@H](C)C(=O)N[C@@H](C)C(=O)N[C@@H](C)C(=O)N[C@@H](C)C(=O)N[C@@H](C)C(=O)N[C@@H](CCCCN)C(=O)O] |
| Tetrapeptide | 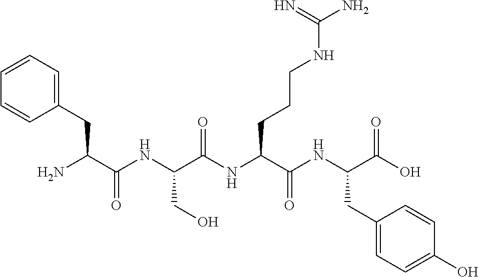 [SMILES: N=C(N)NCCC[C@H](NC(=O)[C@H](CO)NC(=O)[C@@H](N)Cc1ccccc1)C(=O)N[C@@H](Cc1ccc(O)cc1)C(=O)O] |

Tables 1, 2 and 3 are representative, rather than exclusive. Other self-assembling peptides can be also be generated, that differ from those listed in Tables 1-3, for example by replacing a single or multiple amino acids, by inclusion or exclusion of a repeating amino acid, or by incorporating one or more amino acids such that the non-covalent bonding may be increased or decreased and therefore, modify the self-assembled macrostructure within the skin. All such modifications and permutations or combinations are within the scope of the present disclosure.

In order to form a 3D macromolecular structures in vivo within the skin, in certain compositions, the self-assembling peptides are structurally compatible. For example, the side chains (amino acid R groups) of the self-assembling peptides, having no charge (neutral) at physiological pH, for example glycine or valine may be provided. Such self-assembling peptides may have a neutral charge are neutral or may comprise amino acids with no charge at physiological pH. If the charged or neutral amino acids in self-assembling peptides are substituted with a like charge, there are no known significant effects on the self-assembly process. For example, neutral charged valine at position 1 can be replaced by isoleucine. However, if the neutral charged residues are replaced by negatively charged residues (such as, aspartate and glutamate), the self-assembling peptides may be affected. Other amino acids that form hydrogen bonds, such as asparagine and glutamine, may be incorporated into the self-assembling peptides instead of, or in addition to, charged residues. By changing the alanine residues in the self-assembling peptides to more hydrophobic residues, such as leucine, isoleucine, the resulting self-assembling peptides have a greater tendency to self-assemble and form self-assembling peptide matrices with enhanced strength. Some self-assembling peptides that have similar amino acids compositions and lengths as the self-assembling peptides described herein also form other structures. Thus, in addition to structural compatibility other factors can determine the formation of macroscopic matrices, such as the self-assembling peptide length, the degree of intermolecular interaction, and the ability to form staggered arrays etc. Peptides can self-assemble in an unmodified state where the N-terminus of the peptide presents as an amino group of the terminal residue (—NH₂) and the C-terminus of the peptide presents as a carboxylic acid of the opposing terminal residue (—COOH). However, it is possible for peptides to self-assemble when either one or both termini are modified. The charge at the N-terminus, C-terminus, or both can also be modified.

In Table 1, le is denoted as the N-terminus and R 2 is denoted as the C-terminus. There are modifications that traditionally considered N-terminal modifications (R¹), while others are typically C-terminal modifications (R²), and in some instances, the modification could appear on either terminus (R¹=R²). Typical N-terminal modifications (R′) include but are not limited to 5-carboxyfluoroscein (5-FAM), 5-carboxyfluoroscein aminohexanoic acid (5-FAM-Ahx), aminobenzoic acid (Abz), acetyl, acryl. benzoyl, biotin, biotin aminohexanoic acid (biotin-Ahx), tert-butyloxycarbonyl (BOC), bromoacetyl (Br—Ac—), bovine serum albumin (BSA; NH₂ of N-terminal), carboxybenzyl (CBZ), 5-(dimethylamino)naphthalene-1-sulfonyl (Dansyl), 5-(dimethylamino)naphthalene-1-sulfonyl aminohexanoic acid (Dansyl-Ahx), decanoic acid, diethylenetriaminepentaacetic acid (DTPA), fatty acids, fluorescein isothiocyanate (FITC), fluorescein isothiocyanate aminohexanoic acid (FITC-Ahx), fluorenylmethyloxycarbonyl (Fmoc), formylation, hexanoic acid, hydrazinonicotinic acid (HYNIC), keyhole limpet hemocyanin (KLH; NH₂ of N-terminal), lauric acid, lipoic acid, maleimide, 7-methoxycoumarin-4-acetic acid (MCA), myristoyl, octanoic acid, palmitoyl, polyethylene glycol (PEG), stearic acid, and succinylation. Modifications that are traditionally considered C-terminal (R 2) include, but are limited to 7-amino-4-(trifluormethyl)-2-benzopyrone (AFC), 7-amino-4-methylcoumarinyl (AMC), amidation, bovine serum albumin (BSA; —COOH of C-terminal), benzyl (Bzl), cysteamide, ester (OEt, ester (OMe), ester (OtBu), ester (OTBzI), keyhole limpet hemocyanin (KLH; —COOH of C-terminal), multiple antigenetic peptides (MAPs asymmetric 2 branches, MAPS asymmetric 4 branches, MAPS asymmetric 8 branches), methyl (Me), ethylamide (NHEt), isoamyl amido (NHisopen), N-methyl (NHMe), hydroxysuccinimide ester (OSU), ovalbumin (OVA; —COOH of C-terminal), p-Nitroanilide (pNA), and tert-Butyl (tBu).

Further, the peptide may comprise at least one terminal amino acid that has been modified with a non-amino acid organic functional group. Exemplary non-amino acid organic functional groups include, but are not limited to, an alkyl group, an acyl group, a carbohydrate, a polyether, a phosphate, and a fatty acid. In further compositions where the non-amino acid functional group is a phosphate, the phosphate is optionally farnesyl pyrophosphate, geranyl pyrophosphate or 3-isopentenyl pyrophosphate. For example, le may be a group or radical such as an acyl group (R¹CO—, where R¹ is an organic group such as, an acetyl group (CH₃CO—)) can be present at the N terminus of a self-assembling peptide to neutralize an “extra” positive charge that may otherwise be present, such as a charge not resulting from the side chain of the N-terminus amino acid. Similarly, a group such as an amine group (NH₂) in the form of an amide (CO—NH₂) can be used to neutralize an “extra” negative charge that may otherwise be present at the C-terminus, such as a charge not resulting from the side chain of the C-terminus amino acid. The neutralization of charges on a terminus may promote the formation of matrices through self-assembly.

The N-terminus may include an aliphatic group, including a palmitoyl chain of aliphatic acids, with C₁₋₁₀₀, preferably C₁₀₋₂₀ and more preferably, C₁₋₁₆. The N-terminus may include a palmitoyl chain (i.e., C₁₆). Derivatives of the peptides described herein are considered to be encompassed in the present disclosure. Derivatives further include, without limitation, acyl derivatives, with one or more straight chain or branched chain, long or short, saturated or unsaturated, substituted with a hydroxy, amino, amino acyl, sulfate, or sulfide group or unsubstituted having from 1 to 30 carbon atoms. N-acyl-derivatives include those acyl groups which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. These may be substituted or unsubstituted. Preferred embodiments include N-palmitoyl as shown in, SEQ.ID.NO.:3, SEQ.ID.NO.:8, a variation of SEQ.ID.NO.:1 having a pal group (also shown in Table 2) and sequence number #3 having a peptide of sequence GH and its derivatives.

The number of amino acids in self-assembling peptides can vary. The self-assembling peptides may comprise between about 1 amino acid to about 200 amino acids, about 1 to about 36 amino acids, or about 1 to about 16 amino acids or 1 to about 6 amino acids, including all ranges and subranges within. In addition, amino acids may be analogs, D-forms or amino acids that were altered or derivatized by the addition of a chemical group including but not limited to acyl, alkyl, phosphate, farnesyl, isofarnesyl, palmitoyl or fatty acid, etc. Further, the amino acids of the self-assembling peptides can be naturally occurring or non-naturally occurring amino acids. All of the naturally occurring amino acids can be utilized according to the invention in the present disclosure.

In order to enhance bioavailability, the epithelial barrier-crossing properties of those peptides can be improved by increasing their lipophilicity or lipophilic character either by acylation of the N-terminal NH₂ group of the peptide or by esterification of the carboxyl group with an alcohol, linear or branched, saturated or unsaturated, hydroxylated or not, or both. In some embodiments, N-acyl groups used to modify the peptide backbone of the peptide include, but are not limited to, lauroyl (C₁₂), myristoyl (C₁₄), palmitoyl (C₁₆), stearoyl (C₁₈), oleoyl (C_{18:1}), arachidic (C₂₀) or linoleoyl (C_{18:2}) groups. Biotinyl groups (Biotin or derivatives) are also contemplated. The N-terminal group may be either an H or a palmitoyl group.

The ease of penetration or penetration efficiency can also be modulated by incorporating protease or peptidase cleavage sites into the precursors that subsequently form a given structure and by modifying the number of amino acids or the type of amino acid. Proteases or peptidases that occur naturally in vivo may be introduced. Combinations of any of the modifications described herein are within the scope of the present invention. For example, self-assembling peptides that include a protease cleavage site and a cysteine residue and/or a cross-linking agent, kits and devices containing them, and methods of using them can be utilized and are considered to be within the present disclosure.

### Exemplary Peptide Sequence(s)

Classes of self-assembling peptides each with characteristics that define the primary sequence of the molecule are encompassed in the disclosure and are described below. These classes of peptides include, without limiting, dipeptides, surfactant-like peptides, peptide amphiphiles with an alkyl group, bolaamphiphilic peptides, ionic-complementary self-assembling peptides, and cyclic peptides, and the properties that distinguish each class of self-assembling peptides are described herein.

### 1. Dipeptides:

Dipeptides are self-assembling peptides. They are comprised of two amino acids, which can be modified or unmodified. Dipeptides often include aromatic residues, such as phenylalanine, where π-π interactions and the hydrophobic effect strongly influence self-assembly.

### 2. Surfactant-like Peptides and Peptide Amphiphiles:

Surfactant-like peptides are comprised of amino acids having a charged polar amino acid as the head and a repeated sequence of hydrophobic amino acids as the hydrophobic tail. SEQ.ID.NO.:7 is an example of a surfactant-like peptide. The polar head is comprised of a lysine residue and the hydrophobic tail is comprised of six consecutively repeating alanine residues.

Peptide amphiphiles with an alkyl group, on the other hand, include two primary characteristics, primarily, an alkyl tail linked to the N- or C-terminus and a hydrophilic section of amino acids. Secondarily, the sequence may include glycine residues to impart flexibility. The alkyl groups drive the hydrophobic interactions, whereas the polar charged groups prefer to interact with the aqueous environment similar to protein folding.

Non-limiting examples of peptide amphiphile are sequence number #3 (in Table 1) and SEQ.ID.NO.:3. For example, sequence number #3, also shown in Table 2 is a Pal-GH, where the polar head is comprised of the glycine and histidine residues. The histidine is positively charged, and the hydrophobic tail is comprised of a palmitoyl chain conjugated to a glycine residue. In SEQ.ID.NO.:3, which is the Pal-KTTKS, the polar head is comprised of several residues, i.e., KTTKS. The serine and threonine residues are neutral residues and lysine is positively charged head the hydrophobic tail is comprised of a palmitoyl chain.

### 3. Bolaamphiphilic Peptides:

Bolaamphiphilic peptides comprise two hydrophilic heads connected by a region of hydrophobic residues. Asymmetric bolaamphiphiles include differing hydrophilic heads at either end of the hydrophobic region.

### 4. Ionic-complementary Self-assembling Peptides:

Ionic-complementary self-assembling peptides are identified by a hydrophobic tail to engage the hydrophobic effect. The hydrophilic tail comprises charged amino acids to form ionic bonds, and pattern of ionic charges classified into four subtypes. The subtypes of the ionic charges are indicated as: Type I as “+−+−+−,” Type II as “++−−++−−,” Type III as “+++−−−+++,” and Type IV as “+++−−−−.” Non-limiting examples include SEQ.ID.NO.:4, RADA-16, SEQ.ID.NO.:5, IEIK-13, and SEQ.ID.NO.:6, KLD-12, all of which are examples of Type I ionic-complementary self-assembling peptides.

### 5. Cyclic Peptides:

Cyclic peptides may possess an even number of D and L amino acids, which stack via hydrogen bonding, or they can be more amphiphilic in nature and aggregate via the hydrophobic effect.

Each of the non-limiting examples herein describes the self-assembling characteristics of the self-assembling peptides. Peptides described herein are non-limiting and may have the self-assembling characteristics, even if they do not fall within one or more peptide classes described herein. Such examples include SEQ.ID.NO.:1 and its variations and derivatives, SEQ.ID.NO.:2 and SEQ.ID.NO.:8; however, SEQ.ID.NOs.:1-2 are known to self-assemble due to their elastin-like peptide (ELP) sequences. Further exemplary peptides include peptides derived from collagen, variations and derivations of elastin, collagen or self-assembling peptides, peptides from natural protein systems such as fibronectin etc. The applicants demonstrated that the self-assembly of such peptides can be controlled or modified, i.e., prevented from assembly in the formulation while enabling self-assembly by the trigger of choice within the layers of skin. Notably, peptide sequences which do not typically fall within the self-assembly principles or peptide classes yet have the characteristics that help self-assembly and enables self-assembly in vivo, are also encompassed in the disclosure (i.e., SEQ.ID.NO.8). The applicants demonstrated the methods and compositions that prevent assembly of peptides in the formulation, while regulating and effectuating self-assembly via trigger within the layers of skin in vivo, without cellular or tissue biogenesis.

Further, self-assembly materials can include, but are not limited to peptides, proteins, peptoids, lipids, polysaccharides, polynucleotides, aptamers, ligand/receptor couples, and combinations of any of these. Furthermore, choice of oligomers of peptides include structures defined by specific substitutions or sequences that drive specific properties of the networks such as triggering of assembly, delivery through topical application and targeting in tissue, speed, size or shape of network, interaction with skin functional pathways, etc.

### B. Self-Assembling Structures in the Formulation

The compositions described herein, regardless of the precise form and regardless of the overall compositions (e.g., without limitation, whether combined with another agent, within a device, or packaged in a kit or combined with another composition or form etc.) include self-assembly peptides. Particularly, the compositions include peptides that can self-assemble upon a trigger and more particularly, the compositions include peptides SEQ.ID.NOs.:1-8 and sequence numbers #s 1-9, individually or as a mixture. The compositions or mixtures can include various lengths of the same peptide sequence or mixtures thereof or different peptides. For example, self-assembled structures disclosed herein can be formed of individual peptides or heterogeneous mixtures of peptides (i.e., mixtures containing more than one type of peptide conforming to a given formula or to two or more of the formulas).

It is the objective of the present application that when the peptides are present in a formulation, self-assembly is substantially prevented from occurring in the composition prior to the topical application or in vitro. One or more peptides or mixtures thereof, would self-assemble in vivo (i.e., peptides in the mixture are complementary and structurally compatible with each other) upon application of the composition with a trigger that is provided by the treatment or conditions of a trigger, within the skin (i.e., within the epidermal or dermal layers of the skin).

According to the present disclosure, the influence of pH, temperature, hydrophobicity, hydrophilicity or ionic strength of the specific peptide in the composition must be critically managed to effect the sol-gel transition prior to application or treatment of the peptide. In certain formulations, sugars such as, without limiting, sucrose, fructose etc., may also influence the sol-gel transition parameters of the self-assembly material. Upon effecting the sol-gel transition parameters, self-assembly is substantially prevented from occurring in the composition prior to the treatment, while the critical gel concentration is maintained in the composition. For example, the formulation may be free of ions such as monovalent or divalent ions or include very less amounts of ions, monovalent or divalent ions in a range of about 5 M to 0.001 M, including about 2, 0.1, 0.001 M, so that the self-assembly of peptides is substantially prevented from occurring on the external skin. In compositions, the ions may be the salt of mono or divalent cations including but not limited to Na⁺, K⁺, Zn²⁺, ca²⁺, mn²⁺, Fe²⁺, cu²⁺, etc. In compositions, the pH is between about 3 to 9, preferably between about 3 to 7. In some compositions, the amount of peptide is in a range of about 0.001% to 30% by the total weight of the composition, preferably about 0.1% to 10% (w/w) of the total weight of the composition and the temperature is in a range of about 19° C. to 42° C.

C. Self-Assembling Structures in vivo

The compositions comprising self-assembling molecules form macromolecular structures within the skin layers. As described above, the self-assembly of peptides is substantially prevented from occurring on the external skin or in the formulation, however the self-assembly occurs within the layers of the skin subsequent to the delivery of the peptides.

For example, Pal-VGVAPG self assembles within the skin layers into elongated fibers under appropriate triggers in the skin layers such as pH trigger 7, temperature trigger of 37° C. and concentration trigger of >0.01% w/w. Pal-KTTKS, on the other hand, although can self-assemble in vitro (such assembly is substantially prevented from occurring), is triggered by pH of 5 within the skin layers at concentration as low as to 0.5% w/w and the temperature of 37° C. However, increasing the temperature from the composition to skin layers and from the skin surface to the skin layers in a gradient of about 10° C. to 20° C. aids the kinetics of self-assembling within the skin layers, thereby providing a gradient for the trigger to occur at that temperature range. This results in an observable effectuated effect on bulk mechanical properties of the skin. Self-assembly may be initiated or enhanced at any subsequent time following topical application by triggers. The trigger may include an ionic solute or diluent to a peptide composition or a gradient condition within the skin or a change in the value of pH, temperature, among others. Alternatively, addition of salt, such as ZnCl₂ at a concentration of between approximately 0.1 mM and 5 M will induce the assembly within a short period of time (e.g., within a few minutes). Lower concentrations of salt may also induce assembly at a slower rate.

In certain compositions, self-assembly begins when the composition is exposed within the layers of the skin and may be facilitated by the local application of another composition to the area where the composition has been deposited. Based on studies to date, self-assembly also occurs rapidly upon contact with internal bodily tissues. For example, co-assembling of elastin-like peptide onto the pre-existing fibers occurs when a formulation comprising elastin like peptides are administered topically on the skin. The subject, described herein, is a human.

The time required for effective assembly can be 60 seconds or less following contact with a subject's internal tissues, gradients conditions within the skin layers, changes in value of conditions or parameters for such triggers or conditions within the skin that enable such assembly, for example, conditions within the skin layers or found within the body (e.g., in 50, 40, 30, 20, or 10 seconds or less). In some circumstances, where the concentration of self-assembling agents in the composition is low or where the movement of the bodily substance is substantial, self-assembly may take longer to achieve the desired effect, for example, up to a minute, 5 minutes, 10 minutes, 30 minutes, an hour or two or three, or longer. For example, the composition comprising self-assembling peptides when applied to the skin, and subsequently delivered to the layers within the skin, may assembly and form physical functional macrostructures within times as short as 10 seconds following application or as along as 120 minutes following the application.

Individual components or ingredients present in the composition that form a new pattern or self-assembled structure may require additional components or biological molecules that is either available within the skin. A non-limiting example is elastin molecule that exist in the skin layers that aids further assembly within the skin. The disclosure recognizes and defines such assembly of molecules in vivo as co-assembly and the initial structures are referred to as “seed structures”. For example, peptides upon delivery within the skin, may recognize another molecule or a structure within the skin such as, without limiting, elastin, collagen, fibronectin, hyaluronic acid, lipid bilayers, among others and may co-assemble with such molecules creating aggregation and spontaneous assembling, thereby resulting in a bulk physical structure and the resulting cosmetic effect or benefit. In certain instances, such assembly will not require a trigger of choice while the existing seed structures themselves act to facilitate the assembly within the skin layers.

In specific instances, a single class of oligomer of peptides or a single type of oligomer of peptide may self-assemble onto the seed structures. In other instances, two or more different classes or types of oligomer of peptides may assemble with each other and aggregate onto the seed structures within the skin layers or in the tissue. In yet other instances, multiple classes of oligomer of peptides or multiple types of oligomers may co-aggregate with the seed structures within skin layers or with the tissue. Seed structures or molecules include, without limiting, elastin, collagen, and other extracellular molecules (ECM), among others.

In other specific instances, a single class of oligomer of peptides or a single type of oligomer of peptides or multiple classes of oligomer of peptides may self-assemble with another within the skin layers. Such oligomers may not naturally assemble or aggregate with itself or by itself, except that they begin to co-aggregate when they find the other suitable oligomer and aggregates with such oligomer(s) within the skin layers or in the presence of certain in vivo conditions. Further, such oligomers can be designed to assemble only in the presence of another suitable oligomer.

Therefore, the self-assembly occurs where materials such as molecules, polymers, colloids, microscopic or macroscopic particles, delivered within the skin in a specific formulation organizes into ordered and functional structures or patterns as a consequence of specific, local interactions of the components themselves with or without any other driving force. Materials assemble only upon a trigger within the layers of the skin (i.e., within the stratum corneum, epidermis or dermis layers).

Compositions may be administered such that the materials, including peptides are held in a formulation that substantially prevents assembly. However, after the delivery of the material, self-assembly or phase transition is triggered by components found in a subject's body (e.g., ions, seed structures or biological molecules present in the layers of the skin), by physiological pH, temperatures etc., or upon initiation of a trigger.

D. Triggers

The self-assembly systems may employ specific triggers to initiate network formation. As used herein, the term “trigger” means a condition which facilitates or inhibits formation of self-assembly of a particular polymer. A variety of triggers may be utilized in the present disclosure, including, for example:
i. Utilizing “seed structures” in the skin to promote self-assembly. Such seed structures act as a trigger and may include, for example, without limiting elastin fibers, collagen, fibronectin, hyaluronic acid or other existing molecules within the skin layers.
ii. Physical triggers in the composition such as, without limiting, temperature, shear, salinity, concentration, pH, enzyme amounts, light, or ionic concentration, oxidation-reduction potential, hydrophobic/hydrophilic balance, temperature, light, enzyme concentrations and other parameters influencing network formation.
iii. A change in the value of the physical triggers described above between the non-physiological value and the physiological value, including, without limiting, a change in the value of temperature, shear, salinity, concentration, pH, ionic concentration, oxidation-reduction potential, hydrophobic/hydrophilic balance, light, enzyme concentrations and other parameters influencing network formation. Such a change is due to the difference in the value of the respective parameters in the composition and in vivo.
iv. Gradient conditions that exist in the skin layers, including, skin gradient of pH, salinity, ionic strength, enzyme concentrations, among others.
v. Conditions that exists in the composition, for example, the pH of the composition, temperature, salinity, concentration of peptides.
vi. Any combinations of above described triggers.

In methods and compositions, the trigger of temperature in the composition is in a range from about 19° C. to about 42° C. The pH trigger of the composition is in a range of pH 3 to 9. The ionic strength of the composition that may trigger self-assembly is about 0.0001 M to about 5 M. The peptide concentration trigger in the composition ranges from about 0.001% to about 30%.

The change in the temperature value between the composition and skin layer may range from about 1° C. to about 30° C. The change in the concentration within the skin after application is in a range of about 0.001% to about 30% (w/w). The change in the pH value between the pH of the composition and that of the skin layer is in a value range of about 0.0001 to about 5 pH units. The change in the ionic strength value is in a range of about 0.001 M to about 5 M. Self-assembly may be initiated through ionic strength gradient within the skin. For example, monovalent cations such as, Li⁺, Na⁺, K⁺, and Cs⁺, and the concentration of such ions required to induce or enhance self-assembly is typically in a range of about 1 mM to about 5 M. Lower concentrations facilitate assembly, though at a reduced rate wherein higher concentrations may enhance self-assembly.

The pH trigger within the skin is in a range of about 4 to 9. Temperature trigger within the skin is about 35° C. to about 42° C. The peptide concentration trigger within the skin layers is about to about 30% (w/w). The ionic strength trigger within the skin layers is about 0 M to about M.

Similarly, the pH gradient that is available in the various skin layers is a pH of 3 to 7 while the temperature gradient is about 19° C. to about 42° C. All ranges and sub-ranges are included herein.

Self-assembly may be initiated through the salt concentration of the composition, such that when a gradient of the salt concentration exists, the peptides begin to assemble within the skin layers. In some other instances, when a change in the value of salinity exists between the composition and the skin layer, the peptides begin to aggregate and assemble within the skin. Self-assembly can also be initiated by a combination of various triggers described in i-v above.

SEQ.ID.NO.: 1 and its variations and derivatives, for example, self-assemble upon triggers, including and without limitation, gradient of pH, temperature, concentration and ionic strength. The change in the pH value for self-assembly may be between about 1 to about 5. The change in the value of temperature for self-assembly includes about 1° C. to about 30° C., including 1° C., 2° C., 3° C., 4° C., 5° C., 8° C., 10° C., and 20° C. All ranges and sub-ranges are considered to be within the scope of the disclosure. Concentration includes all ranges and sub-ranges. The temperature change is the difference between the ambient temperature (i.e., about 19° C. to about 25° C.) in the formulation prior to the application of the cosmetic composition and the temperature within the skin layers or body temperature of about 35° C. to about 42° C.

For example, SEQ.ID.NO.:1 and its variation with a palmitoyl group, when topically administered in a formulation and is delivered within the skin layers, assembles into elongated fibers of elastin upon triggers as appropriate in the right conditions. Such assembly within the skin provides physical or mechanical changes to the skin, as well as provides structural changes to the skin. Changes may be spontaneous and may be evident upon topical administration of the formulation. Changes in vivo may take place subsequent to the administration of the formulation in at least about 30 seconds to at least about 120 minutes. The physical or mechanical changes may last for about 15 minutes to about 12 hours, or in some case a day or two. The physical and mechanical changes that occur upon self-assembly or co-assembly of molecules include, without limitation, lifting of the skin, enhanced presence of elastin, plumping of the skin, such that the effects are observable. These effects may be caused due to hydrophobic propensity and hydrogen bonding between the molecules within the skin, in specific conditions such that self-assembly is favored.

Specifically, palmitoyl hexapeptide, in presence of an appropriate trigger such as a change in the value of salinity, concentration, pH, and temperature within the skin layers compared to the composition, which forms small fibers creating observable effect on the skin. Under appropriate conditions, the peptide when topically administered and delivered within the skin, in presence of the trigger, co-assembles onto endogenous elastin present within the skin. In the case of palmitoyl hexapeptide, trigger includes, without limitation, a gradient of temperature, pH and salt concentration. For example, the value of temperature change for self-assembly from about 1° C. to about 30° C. modifies the self-assembling of the peptide within the skin. In some other instances, a change in the pH value ranges from about 0.001 to 5 in pH units may be utilized and ionic strength gradient of about 0 to about 5 M ZnCl₂ may be utilized.

SEQ.ID.NO.:2 presents a bulk mechanical effect on the skin when topically administered in an appropriate formulation, and the peptide self assembles within the skin when delivered within the skin. Due to the geometry of sequence number #3 and the ratio of the head to the tail of the peptide, it is most likely that the curvature is not favorable to form long fibers. Therefore, an addition of a fatty acid could potentially trigger increase the length of the fibers by inserting itself within the fiber structure and modifying its geometry to form longer fibers. Such co-mixed fibers then reach a critical length after the entanglement point, and then positively effect the viscoelastic properties.

E. Choice of Self-Assembly Oligomers

In order to impart an effective skin care benefit, selection of self-assembly polymers suitable for delivery is important. Polymers capable of self-assembly may be selected based on a variety of factors. Such self-assembly polymers may include, but are not limited to, peptides described herein. Other polymers include, without limiting, proteins, peptoids, lipids, polysaccharides, polynucleotides, aptamers, ligand/receptor couples, and combinations of thereof. Criterion for selection of oligomers may include choosing structures defined or modified by specific substitutions or sequences that drive specific properties of the networks, such as triggering of assembly, delivery and targeting in tissue, speed, size or shape of network, interaction with skin functional pathways, etc. Preferred oligomers may be optimized in view of in silico modeling and design and testing on skin models.

### Cosmetic Compositions

The present disclosure provides cosmetic compositions comprising self-assembly materials, including peptides. The self-assembly materials and the cosmetic compositions comprising such materials are administered topically on the human skin and thereby are delivered within the layers of the skin forming macrostructures within the skin providing physical and visible cosmetic benefit on the skin. The compositions improve mechanical and physical characteristics of the skin without biogenesis of cells or tissues. The compositions may be applied at a suitable amount, weight or concentration effective to provide the cosmetic benefit. The compositions may be formulated in various forms and amounts for ease of administration and to achieve the desired effect.

Various product forms and formulation types are contemplated in the present invention, including gels, membranes, powders, sprays, films, liquids, creams, foams, emulsions, masks, watery lotions, watery creams. These forms are also relevant to cosmetic and dermatological applications, as are masks, patches, applicators.

Formulation parameters that effect macrostructure assembly formation within the skin include among other things, pH, salt and peptide concentration. The self-assembling or lack thereof, of the oligopeptide after application to the skin may take place because of changes or gradient of the pH, temperature, peptide concentration or salt concentration triggered by the application of the formulation. More specifically, in the epidermal layers of the skin, pH (approximate value of 5.5) can alter the formulation pH, allowing oligopeptide self-assembling. Other mechanisms of initiation can include pH, salt concentration, and oligopeptide concentration changes or its gradient via evaporation of the carrier. Self-assembling peptides can be delivered with a hydrophobic material (e.g., a cosmetically acceptable oil, oil in water or emulsion). The self-assembling peptides may be mixed with a hydrophobic agent such as an oil or lipid or may exist in an emulsion or oil-in-water form.

According to the present invention, formulations and packages must be designed to substantially prevent self-assembly from occurring prematurely in the composition and prior to the application or treatment. Furthermore, the formulations must help drive specific triggers including: triggers provided by the personal care procedure/regime including a. changes in shear, evaporation, temperature changes, dilution or concentration, mixing of multiple phases and/or mixing of multiple complementary oligomers during use; and, b. changes in physical parameters (driven either in the formulation/packaging itself or through the differences in skin vs the formulation) such as pH, ionic strength, hydrophobic/hydrophilic balance, temperature, and other parameters influencing network formation. Also, the formulation must be designed to deliver or promote penetration of the self-assembly oligomers to the right layer in or on the skin or scalp.

The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to a composition herein. Examples of these ingredient classes include, but are not limited to: abrasives, absorbents, aesthetic components such as fragrances, pigments, colorings/colorants, essential oils, skin sensates, astringents, such as cosmetic and drug astringents (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antibacterial agents, antifungal agents, antioxidants, binders, biological additives, buffering agents, bulking agents, chelating agents, chemical additives, colorants, cosmetic biocides, denaturants, external analgesics, film formers or materials, e.g., polymers, for aiding the film-forming properties and substantivity of the composition (e.g., copolymer of eicosene and vinyl pyrrolidone), opacifying agents, pH adjusters, plant derivatives, plant extracts, plant tissue extracts, plant seed extracts, plant oils, preservatives, propellants, reducing agents, sebum control agents, and sequestrants. The concentration of self-assembling peptides can vary from approximately 0.001% w/v (0.001 mg/mL) to approximately 99.99% w/v (999.9 mg/mL), inclusive. For example, the concentration in the formulation can be between approximately 0.001% (0.1 mg/mL) and 30% (3000 mg/mL), inclusive (e.g., about 0.01%-5%; 0.5%-5%; 1.0%; 1.5%; 2.0%; 2.5%; 3.0%; or 4.0% or 10%, 20% or more). In some embodiments, the concentration can also be less than 0.001%. The concentration of the self-assembling materials, such as peptides in any given formulation can vary and can be between approximately 0.001% and 30%, inclusive, preferably between 0.001% and 30% by weight of the total weight of the composition. The concentration of the self-assembling materials (e.g., in a liquid, serum, cream, gel formulation) can be approximately 0.001-30.0% (0.1-300 mg/mL) (e.g., 0.01-10%; 1.0-20.0%; 2.0-30.0% or 0.5-30.0% by weight of the total weight of the composition, including all ranges and subranges). The concentration of self-assembling materials can be higher in stock solutions and in solid (e.g., powdered) formulations. Solid preparations may have a concentration of self-assembling materials approaching 100% by weight (e.g., the concentration of self-assembling materials can be 95, 96, 97, 98, 99% or more (e.g., 99.99%) by weight of the composition). Whether in liquid or solid form, the materials such as peptides can be brought to the desired concentration by addition of an acceptable diluent (e.g., water), fillers, or oil. The formulations may include an acceptable carrier or other agents. The amount of self-assembling peptide, for example, in composition comprising at least one of SEQ.ID.NOs.:1-8, sequence numbers #1-9, may be in an amount ranging from about 0.001% to about 30% (w/w) by the total weight of the composition. In certain compositions, the amount of self-assembling peptide is about to about 15% (w/w) by the total weight of the composition. In certain compositions, the amount of self-assembling peptide is about 0.5% to about 20% (w/w) by the total weight of the composition. In certain compositions, the amount of self-assembling peptide is about 0.5% to about 10% (w/w) by the total weight of the composition. All ranges and sub-ranges are considered to be encompassed herein.

The composition disclosed herein further may comprise a biological molecule, including, without limiting, as a protein, peptide or bio-organic molecule in addition to the peptides. The biological molecule may be mixed with the self-assembly materials disclosed herein. Such biological molecules may be provided as a separate solution and may be applied prior to or following the topical application of the composition. The composition may also comprise additional unmodified peptides that are structurally complementary and compatible and allows for increased cosmetic benefit on the skin. Such additional peptides in the composition and the peptides disclosed herein is present in a ratio of about 1:1, 1:10, 1: 100, 10: 1, 5:1, 9:1 or 99:1 or in subranges within.

The compositions are substantially free of a gelation agent. This enables self-assembly to occur within the body to form a macroscopic structure (i.e., within the skin layers upon contact with tissue).

Other optional components which may be incorporated in a composition described herein include, but are not limited to, one or more cosmetic skin care agents. A cosmetic skin care agent is any substance, material, or compound, intended to be applied to the skin for the purpose of improving an undesirable skin condition (or symptom thereof). Some undesirable skin conditions include outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles, including both fine superficial wrinkles and coarse deep wrinkles, folds, skin lines, crevices, bumps, large pores (e.g., associated with adnexal structures such as sweat gland ducts, sebaceous glands, or hair follicles), scaliness, flakiness and/or other forms of skin unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

The treatment regimens/procedures required for the self-assembly networks to form must provide control over several possible parameters including proximity of interacting oligomers, concentration of oligomers, in use shear forces, layering of interacting phases, enhanced penetration of oligomers, targeting of networks to specific places on or in skin, and triggering through changes in physical parameters.

The composition may comprise a dermatologically, cosmetically acceptable carrier or excipient. The carrier may thus act as a diluent, dispersant, solvent, or the like for the peptide and other materials, compounds and/or agents. Exemplary acceptable excipients include any and all solvents, dispersion media, diluents, or other liquid vehicles, dispersion or suspension aids, surface active agents, isotonic agents, thickening or emulsifying agents, preservatives, solid binders, lubricants as suited for the topical administration and dosage. Except insofar as any conventional carrier medium is incompatible with a substance or its derivatives, such as by producing any undesirable biological effect or otherwise interacting in a deleterious manner with any other component(s) of the composition, its use is contemplated to be within the scope of this invention. The carrier may contain one or more acceptable solid, semi-solid or liquid fillers, diluents, solvents, extenders and the like. The carrier may be solid, semi-solid or liquid. The carrier can itself be inert or it can possess dermatological or cosmeceutical benefits of its own. Concentrations of the carrier can vary with the carrier selected and the intended concentrations of the essential and optional components. In compositions, the carrier is present at a level of from about 50% to about 99.99% (e.g., from about 60% to about 99.9%, or from about 70% to about 98%, or from about 80% to about 95%), by weight of the composition. The acceptable carrier may be provided in a wide variety of forms. Non-limiting examples include, but are not limited to, simple solutions (water or oil-based), emulsions, and solid or semi-solid forms (gels, sticks). For example, emulsion carriers can include, but are not limited to, oil-in-water, water-in-oil, water-in-silicone, water-in-oil-in-water, and oil-in-water-in-silicone emulsions. As will be understood by the skilled artisan, a given component will distribute primarily into either the water or oil phase, depending on the water solubility/dispersibility of the component in the composition. In some embodiments, a personal care composition described herein is formulated into an oil-in-water emulsion.

Suitable carriers also include oils. The composition may comprise from about 1% to about 95% by weight of one or more oils. The composition may comprise from about 0.1%, 0.5%, 1%, 2%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, 5%, or 3% of one or more oils. Oils may be used to solubilize, disperse, or carry materials that are not suitable for water or water soluble solvents. Suitable oils include silicones, hydrocarbons, esters, amides, ethers, and mixtures thereof. Oils may be fluid at room temperature. The oils may be volatile or nonvolatile. “Non-volatile” means a material that exhibit a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm Hg at 20° C. Volatile oils may be used to provide a lighter feel when a heavy, greasy film is undesirable. When the skin care composition is in the form of an emulsion, oils are carriers typically associated with the oil phase. The composition may comprise an emulsifier. An emulsifier is particularly suitable when the composition is in the form of an emulsion or if immiscible materials are being combined. The skin care composition may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. The compositions described herein may be in the form of pourable liquids (under ambient conditions). The compositions can therefore comprise an aqueous carrier, which is typically present at a level of from about 20% to about 95% (or from about 60% to about 85%) based on weight of the composition. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

The compositions of the present invention also comprise, the carrier is present at a level of from about 20% to about 99.99%, about 30% to about 90%, about 40% to about 80% by weight of the composition. The carrier can be in a wide variety of forms. Non-limiting examples include simple solutions (e.g., aqueous, organic solvent, or oil based), emulsions, suspensions, and solid forms (e.g., gels, sticks, flowable solids, or amorphous materials). In certain embodiments, the dermatologically acceptable carrier is in the form of an emulsion or suspension. Emulsions or suspensions may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. Nos. 3,755,560, 4,421,769, and McCutcheon's Detergents and Emulsifiers, North American Edition, pages 317-324 (1986). Suitable emulsions may have a wide range of viscosities, depending on the desired product form. The carrier may further comprise a thickening agent as are well known in the art to provide compositions having a suitable viscosity and rheological character.

The compositions herein may further include at least one film-forming polymer. The film-forming polymer may be chosen from cellulose polymers, such as nitrocellulose, cellulose acetate, cellulose acetate/butyrate, cellulose acetate/propionate, and ethyl cellulose; polyurethanes; acrylic polymers; vinyl polymers; polyvinylbutyrals; alkyd resins; resins resulting from aldehyde condensation products, such as arylsulphonamide-formaldehyde resins, for example, toluenesulphonamide-formaldehyde resin, and arylsulphonamide-epoxy resins. Further non-limiting examples of suitable film-forming polymers include nitrocellulose from Hercules; toluenesulphonamide-formaldehyde resins “Ketjentflex MS80” from Akzo, “Santolite MHP”, “Santolite MS 80”, and “Resimpol 80” from Pan Americana, the alkyd resin “Beckosol ODE 230-70-E” from Dainippon, the acrylic resin “Acryloid B66” from Rohm & Haas, and the polyurethane resin “Trixene PR 4127” from Baxenden. The film-forming polymer may generally be present at about 1% to about 50%, preferably from about 2% to about 40%, and most preferably from about 2% to about 35% of the composition.

### Transdermal Delivery Vehicle

In order to self-assemble beneath the surface of the skin, the self-assembly material herein may optionally be delivered via a transdermal delivery vehicle. The transdermal delivery vehicle may comprise a physical or chemical mechanism designed to deliver materials beneath the skin's surface. Such physical mechanisms may include, for example, known penetration enhancing cosmetic compositions or ingredients, hypodermic needles, microneedles, transdermal patches, electrospun nanofibers, and the like. Other delivery systems may include, for example, using chemical enhancers, non-cavitational ultrasound, iontophoresis and other energy devices. The compositions herein may further include at least one film-forming polymer. The film-forming polymer may be chosen from cellulose polymers, such as nitrocellulose, cellulose acetate, cellulose acetate/butyrate, cellulose acetate/propionate, and ethyl cellulose; polyurethanes; acrylic polymers; vinyl polymers; polyvinylbutyrals; alkyd resins; resins resulting from aldehyde condensation products, such as arylsulphonamide-formaldehyde resins, for example, toluenesulphonamide-formaldehyde resin, and arylsulphonamide-epoxy resins. Further non-limiting examples of suitable film-forming polymers include nitrocellulose from Hercules; toluenesulphonamide-formaldehyde resins “Ketjentflex MS80” from Akzo, “Santolite MHP”, “Santolite MS 80”, and “Resimpol 80” from Pan Americana, the alkyd resin “Beckosol ODE 230-70-E” from Dainippon, the acrylic resin “Acryloid B66” from Rohm & Haas, and the polyurethane resin “Trixene PR 4127” from Baxenden. The film-forming polymer may generally be present at about 1% to about 50%, preferably from about 2% to about 40%, and most preferably from about 2% to about 35% of the composition.

### Adhesive Agents

The compositions of the present invention can comprise from about 0.1% to about 10%, preferably from about 0.1% to about 2% of an adhesive agent. The species and levels of the adhesive agents are selected to provide, for example, a more flexible, longer-lasting benefit to composition, and/or better compatibility with other skin care or cosmetic formulations. Examples of suitable adhesive agents include polyurethanes, including Polyderm PE-PA, available from Alzo International Inc.; co-polymerized amido ester compounds, including Polyderm PPG-17, available from Alzo International Inc.; and acrylic latex dispersions.

### Skin Active Agents

The compositions of the present invention may comprise a skin active agent which provides a particular skin care benefit characteristic of the usage of the skin care product. Herein, skin care benefit may include benefits related to appearance or make-up of the skin. The skin care active can provide acute (immediate and short lived) benefits, or chronic (long term and longer lasting) benefits.

The skin active agents useful herein include skin lightening agents, anti-acne agents, emollients, non-steroidal anti-inflammatory agents, topical anesthetics, artificial tanning agents, anti-microbial and anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, anti-itch agents, hair growth inhibitors, desquamation enzyme enhancers, anti-glycation agents, and mixtures thereof. When included, the present composition comprises from about 0.001% to about 20%, preferably from about 0.1% to about 10% of at least one skin active agent.

The type and amount of skin active agents are selected so that the inclusion of a specific agent does not affect the stability of the composition. For example, hydrophilic agents may be incorporated in an amount soluble in the aqueous phase, while lipophilic agents may be incorporated in an amount soluble in the oil phase.

Other skin active agents purported to exhibit expression-line relaxing benefits for use in the present invention include, but are not limited to, Lavandox available from Barnet Products Corporation; Thallasine 2, available from BiotechMarine; Argireline NP, available from Lipotec; Gatuline In-Tense and Gatuline Expression, available from Gattefosse; Myoxinol LS 9736 from BASF Chemical Company, Syn-ake, available from DSM Nutritional Products, Inc.; and Instensyl®, available from Silab, Inc; Sesaflash™, available from Seppic Inc.

Skin lightening agents useful herein refer to active ingredients that improve hyperpigmentation as compared to pre-treatment. Useful skin lightening agents herein include ascorbic acid compounds, vitamin B3 compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-O-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-O-D-glucopyranosyl-L-ascorbic acid available from Hayashibara and sodium L-ascorbyl phosphate with tradename STAY C available from Roche.

Vitamin B3 compounds useful herein include, for example, those having the formula:

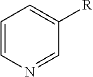

wherein R is —CONH₂ (e.g., niacinamide) or —CH₂OH (e.g., nicotinyl alcohol); derivatives thereof; and salts thereof. Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B3 compounds are niacinamide and tocopherol nicotinate, and more preferred is niacinamide. In a preferred embodiment, the vitamin B3 compound contains a limited amount of the salt form and is more preferably substantially free of salts of a vitamin B3 compound. Preferably the vitamin B3 compound contains less than about 50% of such salt and is more preferably essentially free of the salt form. Commercially available vitamin B3 compounds that are highly useful herein include niacinamide USP available from Reilly.

Other hydrophobic skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); Oenothera biennis seed extract, and pyrus malus (apple) fruit extract, water and Myritol 318 and butylene glycol and tocopherol and ascorbyl tetraisopalmitate and Paraben and Carbopol 980 and DNA/SMARTVECTOR UV available from COLETICA, magnesium ascorbyl phosphate in hyaluronic filling sphere available from COLETICA, and mixtures thereof.

Other skin active agents useful herein include those selected from the group consisting of N-acetyl-D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetaminophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. In a preferred example, the content level of a skin active agent is from about 0.001% to about 20%, more preferably from about 0.1% to about 10%.

### Thickener

Useful for the present invention is a thickener. Thickeners can be used for solidifying solid water-in-oil form compositions of the present invention. When used, the thickener is kept to about 15% or less of the composition. The thickeners useful herein are selected from the group consisting of fatty compounds, gelling agents, inorganic thickeners and mixtures thereof. The amount and type of thickeners are selected according to the desired viscosity and characteristics of the product. These characteristics may include a synergistic effect between the thickener and the film forming ingredients, thereby enhancing product/film adhesion, contraction, or flexibility, while decreasing whiteness.

Thickening agents which can be used in the present invention include, but are not limited to, cross-linked polyacrylates such as Carbopol™ (Goodrich); polyacrylate copolymers such as Sepimax Zen (Seppic, Inc.); modified acrylate copolymers such as Sepiplus S (Seppic, Inc.) polymeric carboxylates including modified and unmodified starches, polysaccharide gums such as xanthan gum (e.g., CP Kelco's Keltrol CGT and Keltrol T630, Jungbunzlauer's Xanthan Gum), dehydroxanthan gum (e.g., Amaze XT from AkzoNobel), galactomannan (Solagum Tara from Seppic), and cellulose derivatives (e.g., Natrosol 250). Gums may also include, but are not limited to, crosslinked-xanthan gum, hydroxypropyl xanthan gum, undecylenoyl xanthan gum, deacetylated xanthan gum, guar gum, cellulose gum, carrageenan, hydroxylpropyl methyl cellulose, and sodium carboxymethyl chitin.

Polymers useful herein include swellable, lightly to moderately crosslinked polyvinylpyrrolidones (PVP) such as ACP-1120 (International Specialty Products), acrylate copolymers/crosspolymers/blends such as acrylate/steareth-20 itaconate copolymer (Structure 2001 from AkzoNobel), acrylates/C₁₀₋₃₀ alkyl acrylates copolymer (Amaze XT from AkzoNobel), acrylic acid/VP crosspolymer (Ultrathix P100 from International Specialty Products).

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

The gelling agent useful as thickeners of the present invention include esters and amides of fatty acid gellants, hydroxy acids, hydroxy fatty acids, other amide gellants, and crystalline gellants. N-acyl amino acid amides useful herein are prepared from glutamic acid, lysine, glutamine, aspartic acid and mixtures thereof.

### Other Optional Components

The compositions hereof may further contain additional components such as those conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits), optically active and color enhancing materials or ingredients, concealers, blurring agents, dyes, pearls, pigments, etc.

These components may include, but are not limited to, materials purported to smooth, firm or lift sagging or wrinkled skin including: Quicklift, available from BASF Chemical Company; Syntran PC5100, available from Interpolymer Corporation; Glycolift, available from Solabia USA Inc.; Alguard, available from Frutarom; Easyliance, from Soliance; and Phytodermina Lifting code 9002, available from Istituto Ricerche Applicate.

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

### Use

Various methods of treatment, application, regulation, or improvement may utilize the aforementioned compositions. Application of the present compositions can occur on any skin surface of the body. Skin surfaces of the most concern tend to be those not typically covered by clothing such as facial skin surfaces, hand and arm skin surfaces, foot and leg skin surfaces, and neck and chest skin surfaces (e.g., décolletage). In particular, application may be on a facial skin surface including the forehead, perioral, chin, periorbital, nose, and/or cheek skin surfaces. The use of the composition disclosed herein improves mechanical and physical characteristics of the skin without biogenesis. This improvement in the mechanical characteristic of the skin provides visible cosmetic benefits and effects disclosed herein through regiments and treatment methods provided herein.

Many regimens exist for the application of the composition to the skin. The composition may be applied at least once a day, twice a day, or on a more frequent daily basis, during a treatment period. When applied twice daily, the first and second applications are separated by at least 1 to about 12 hours. Typically, the composition may be applied in the morning and/or in the evening.

The cosmetic composition may be applied as a treatment regimen that includes other products or formulations. A different composition or a secondary application of a different condition such as temperature, penetration enhancers by, devices or ingredients may be applied prior to or following the application of the composition. Preferably, such applications may be incorporated as a second, third, fourth or further treatment steps. Such applications may condition the skin in advance to the treatment application using the cosmetic composition. For example, the secondary or other application steps can alter the pH of the skin, temperature, salinity, or other conditions that prepare and induce self-assembly within the skin following or prior to the treatment. Any number of products or treatment steps may be included as part of the regimen, in any necessary order. Examples of the uses of the second or different composition includes altering pH, ionic strength, temperature, addition or removal of chemical or biochemical triggers, etc., in advance of the treatment. By using a regimen, or several applications of the self-assembly material, the treatment can allow materials to build up in tissue achieving a critical concentration, and allowing it to see its nearest neighbor, thereby creating 2D and/or 3D macromolecular structures and assemblies. Such repeated use of the treatment methods and compositions described herein will provide accumulation of the self-assembly material and structures within the skin layers, thereby providing enhanced cosmetic benefit with passage of time.

The step of applying the composition to the skin may be done by localized application to an area that contains wrinkles. In reference to application of the composition, the term “localized”, “local”, or “locally” mean that the composition is delivered the targeted area (such as an area of skin containing wrinkles) while minimizing delivery to skin surface or subdermal layers not requiring treatment. The composition may be applied and lightly massaged into the skin. It is recognized that localized application allows for a reasonable amount of the composition to be applied to areas adjacent the wrinkles to be treated (i.e., the composition is unlikely to be applied or to remain within the boundary of the wrinkles without some spreading). The form of the composition or the dermatologically acceptable carrier should be selected to facilitate localized application. While certain embodiments of the present invention contemplate applying a composition locally to a wrinkled area, it will be appreciated that compositions of the present invention can be applied more generally or broadly to one or more facial skin surfaces to reduce the appearance of wrinkles within those facial skin regions. Likewise, the compositions can be applied as a continuous film, or in patterns. Striations, patterned spots or random application of the compositions may be desirable. Applicators, as described below, may be beneficial assisting in patterned deposition.

According to a particular method, the compositions may be applied to skin regions where a desired lifting effect is desired. For example, the composition may be applied to the hairline, temples, jawline, and other peripheral facial regions in order to apply a lifting effect to other facial regions. This method utilizes a lifting effect at the periphery of the face to reduce the appearance of wrinkles at, for example, the eye region, smile lines around the mouth, under-eye wrinkles, and to smooth wrinkles around cheek areas. According to this method, the composition may be applied to the periphery of the face without applying the composition directly to target wrinkles.

In another aspect, the present disclosure provides a method of improving mammalian skin comprising administering an effective amount of a composition. In some embodiments, the improving of mammalian skin comprises treatment of a mammalian keratinous tissue condition. Such treatment of keratinous tissue conditions can include topical application, including improving the cosmetic appearance of the mammalian keratinous tissue. In some embodiments, the method includes, but is not limited to, preventing, retarding, and/or treating uneven skin tone; reducing the size of pores in mammalian skin; regulating oily/shiny appearance of mammalian skin; thickening keratinous tissue (i.e., building the epidermis and/or dermis and/or subcutis layers of the skin and where applicable the keratinous layers of the nail and hair shaft); preventing, retarding, and/or treating uneven skin tone by acting as a lightening agent or a pigmentation reduction cosmetic agent; preventing, retarding, and/or treating atrophy of mammalian skin; softening and/or smoothing lips, hair and nails of a mammal; preventing, retarding, and/or treating itch of mammalian skin; preventing, retarding, and/or treating the appearance of dark under-eye circles and/or puffy eyes; preventing, retarding, and/or treating sallowness of mammalian skin; preventing, retarding, and/or treating sagging (i.e., glycation) of mammalian skin; preventing and/or retarding tanning of mammalian skin; desquamating, exfoliating, and/or increasing turnover in mammalian skin; preventing, retarding, and/or treating hyperpigmentation such as post-inflammatory hyperpigmentation; preventing, retarding, and/or treating the appearance of spider vessels and/or red blotches on mammalian skin; preventing, retarding, and/or treating fine lines and wrinkles of mammalian skin; preventing, retarding, and/or treating skin dryness (i.e., roughness, scaling, flaking); and preventing, retarding, and/or treating the appearance of cellulite in mammalian skin. In some embodiments, the composition is used to treat the signs of aging. For example, in some embodiments, the composition is used to regulate the signs of aging. In some embodiments, the composition is used to reduce or decrease the signs of aging. In some embodiments, the composition is used to prevent the signs of aging in keratinous tissue (e.g., skin, hair, or nails). Improving keratinous tissue conditions can involve topically applying to the keratinous tissue a safe and effective amount of a composition of the present disclosure.

Non-limiting examples of skin care compositions include, but are not limited to, sunscreens and blocks, mousse, bath and shower gels, lip balms, skin conditioners, cold creams, moisturizers, soaps, body scrubs, body wash, face wash, body spray, exfoliants, astringents, scruffing lotion, depilatories shaving, pre-shaving and after-shaving products, deodorants and antiperspirants, cleansers, skin gels, and rinses, skin lightening and self-tanning compositions. Non-limiting examples of hair care compositions include, but are not limited to, shampoo, conditioner, treatment, styling, hair spray, permanent styling, tonics, cream rinse, hair dye, hair coloring, hair bleaching, hair shine, hair serum, anti-frizz, volumizers, split-end repair, anti-dandruff formulations, and mascara. Non-limiting examples of other cosmetic compositions include but are not limited to make up, including lipstick, rouge, foundation, blush, eyeliner, lip liner, lip gloss, facial or body powder, nail polish, eye shadow, among others. Furthermore, the composition can be applied topically through the use of a patch or other delivery device. Delivery devices can include, but are not limited to, those that can be heated or cooled, as well as those that utilize iontophoresis or ultrasound. In some embodiments, for example, the composition described herein is in the form of a skin lotion, clear lotion, milky lotion, cream, gel, foam, ointment, paste, emulsion, spray, conditioner, tonic, cosmetic, lipstick, foundation, nail polish, after-shave, or the like, which is intended to be left on the skin or other keratinous tissue for some aesthetic, prophylactic, therapeutic or other benefit (i.e., a “leave-on” composition or skin care composition). After applying the composition to the keratinous tissue (e.g., skin), it is preferably left on for a period of at least about 2 minutes, 5 minutes, 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.). The application of the present compositions may be done using the palms of the hands and/or fingers or a device or implement (e.g., a cotton ball, swab, pad, applicator pen, spray applicator, etc.).

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows-feet area, frown lines, under-eye area, upper lip, and the like). The patch can be occlusive, semi-occlusive or non-occlusive, and can be adhesive or non-adhesive. The composition can be contained within the patch or be applied to the skin prior to application of the patch. In some embodiments, the patch is formed from the self-assembled peptide structure itself, without the need for an additional, non-peptide substrate. The patch can also include additional actives such as chemical initiators for exothermic reactions. The patch can also contain a source of electrical energy (e.g., a battery) to, for example, increase delivery of the composition and active agents (e.g., iontophoresis). The patch is preferably left on the keratinous tissue for a period of at least about 5 minutes, or at least about 15 minutes, or at least about 30 minutes, or at least about 1 hour, or at night as a form of night therapy.

### Applicators

In some embodiments, the composition may be delivered by a variety of applicators appropriate for localized and general application. By way of example, a suitable applicator may be a dropper and bottle that contains the composition. A pen-like wand with a housing that may contain the composition can also be used. The wand may comprise a handle, a stem, and an applicator head. The applicator head may comprise fibers, foam, cotton, a roller ball or any other suitable material that may releasably hold the composition. Exemplary applicators include devices, penetration enhancers, microneedles, among others.

A simple cotton swab can apply the composition locally to the wrinkled area. Other suitable applicators include SH-0127 pen applicator available from Shya Hsin Plastic Works, Inc., Taiwan and either the Xpress Tip or liquid filled swab available from SwabPlus, Inc., China. The applicator may be configured to easily apply the composition to wrinkled areas having an approximate diameter between about 2 mm and about 20 mm and allowing for a dosed amount of the composition of between about 0.01 to about 2 mg/cm′ or between about 0.1 to about 1 mg/cm′. Thickness of the applied film can be measured or calculated based on the application are and application dose given directly above.

In another embodiment, the applicator may be in the form of a pretreated tape. The tape may be treated or impregnated with the composition herein, then applied to skin via any suitable tape-dispensing mechanism. In a particularly preferred embodiment, the tape may comprise a dissolvable material, such as a water-soluble polyvinyl alcohol (PVA) film. Such a material allows the user to apply the pretreated tape without having to remove the tape backing upon application since the backing would eventually dissolve, leaving only the composition on the treated site.

In a preferred embodiment, the applicator may promote transdermal delivery of the compositions herein. Therefore, the applicator may take the form of a transdermal patch, a microneedle applicator, or the like.

### Kits

As described generally above and herein, the present invention also provides kits comprising the inventive self-assembling peptide or the composition thereof. Kits are typically provided in a suitable container (e.g., for example, a foil, plastic, or cardboard package). An inventive kit may include one or more excipients, additives, and the like, as is described herein. The inventive kit may include means for proper administration including an applicator. The inventive kit may include instructions for proper administration and/or preparation for proper administration. For example, in certain embodiments, the present invention provides a cosmetic kit comprising: (a) an inventive self-assembling peptide, a cosmetic composition thereof; (b) instructions for initiating self-assembly of the peptide into a macroscopic structure within the skin; and (c) instructions for introducing the self-assembling peptide into a subject topically, including in a liquid in which the peptide can be dissolved, an ion or salt thereof for initiating or triggering peptide self-assembly, and one or more optional compositions, triggers or treatment regimens, as required.

While some methods described herein contemplate applying the compositions of the present invention with an applicator, it will be appreciated that applicators are not required, and the compositions of the present invention can also be applied directly by using one's finger or in other conventional manners.

The dimensions and values disclosed herein are not to be understood as being strictly limited to the exact numerical values recited. Instead, unless otherwise specified, each such dimension is intended to mean both the recited value and a functionally equivalent range surrounding that value.

### EXPERIMENTS AND EXAMPLES

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance, which can be adapted to the practice of this invention in its various embodiments and the equivalents thereof.

### Example 1: Exemplary Composition

| Ingredient INCI name | w/w % | Function |
|---|---|---|
| Water | QS | Solvent |
| Disodium EDTA | 0.01-0.09% | Chelating agent |
| Caffeine | 0.01-0.2% | Conditioning agent |
| 1,3-Butylene glycol | 2-5% | Anti-freeze |
| Glycerin | 2-5% | Conditioning agent |
| Xanthan gum | 0.5-2% | Thickener |
| Self-assembling peptide | 0.001-30% | Active |
| Polysorbate-20 | 0-1% | Emulsifier |
| Phenoxyethanol | 0.4-0.9% | Preservative |
| PEG-60 hydrogenated castor oil | 0.1-3% | Solubilizer |
| CaCl₂ | 0-0.5% | Salt |
| Citric acid/Sodium Citrate | 0-0.5% | PH adjustor |

### Test Methods

### Example 2: Characterization of the Self-Assembly Composition

Solution-gel (sol-gel) transition of each peptide was investigated in a study and the results are as shown in Table 4 below. In some cases, where sol-gel transition is impacted by addition of salt, the higher the ionic strength is, the lower the peptide concentration must be to form a gel and the formulation parameters are maintained in the compositions such that the peptides does not form a gel.

TABLE 4

| | | C0 for sol-gel | | | |
|---|---|---|---|---|---|
| | | transition by | | | |
| | | weight of the | Sol-gel | Ionic | |
| | | composition | transition | strength | Temperature |
| Peptide | Name | (w/w) | pH | trigger | trigger |
| RADARADARADARADA | RADA 16 | 2% | 7 | YES | NO |
| AAAAAAK | A6K | greater than 2% | NA | NA | NA |
| KLDLKLDLKLDL | KLD-12 | 2% | 5 | NO | NO |
| IEIKIEIKIEIKI | IEK-13 | 2% | 3 | NO | YES |
| PAL-VGVAPG | Palmitoyl | 1% | 7 | YES | YES |
| | hexapeptide | | | | |
| PAL-KTTKS | Palmitoyl | 0.5% | 5 | NO | YES |
| | pentapeptide-4 | | | | |
| ACE-IGVAPG | Elastin hexapeptide | NA | NA | NA | NA |
| PAL-GH | Palmityl dipeptide | 1.25% | 5 | YES | YES |

In compositions, the influence of pH of 3 to 9, ionic strength of about 0 M to about 5 M salt concentration (for example, ZnCl₂, NaCl etc.) via addition of any monovalent and/or divalent cation or temperature plays a critical role in preventing the self-assembly in formulation. In compositions, the pH maybe in the range of about 3 to 9. In some compositions, the amount of peptide is in a range of about 0.001% to 30% by weight and the temperature of the composition may be in a range of about 19° C. to 42° C.

### Example 3: Temperature Dependent Self-Assembly of Peptides in ex vivo Dermal Tissues

The generation of elastin fibers involves the self-assembly process of coacervation causing an accumulation of hydrophobic tropoelastin molecules prior to being cross-linked at lysines to generate mature elastin fibers. Elastin-like peptides (ELPs) undergo temperature-driven conformational change upon heat referred to as inverse temperature transition (ITT). The inventors of the present application showed self-assembly of peptides to ex vivo dermal tissue, that is devoid of cells, under different temperature conditions indicating that self-assembly occurs on existing elastin fibers present in the layers of the skin and is enhanced with increasing temperatures.

To demonstrate temperature driven self-assembly of ELPs on existing elastin fibers, abdominal skin samples were procured, stabilized overnight in DMEM containing 10% fetal bovine serum (FBS) and 3% penicillin/streptomycin/amphotericin B. Biopsies (12-mm) were isolated and submerged in a de-epthithelization buffer (0.605% Trizma, 4% NaCl and 0.202% of EDTA in PBS) overnight at 37° C. to remove the epidermis, followed by submersion in four changes of a decellularization buffer (1% Triton X-100 and 0.25% tributyl phosphate in PBS) for 48 hours at 37° C. Following decellularization, the skins, here after referred to as dermises, were washed three times in PBS buffer (1X) for 2 hours each. The decellularized dermises were submerged in either PBS, 100 μg/mL Pal-VGVAPG, or Ace-IGVAPG, for five days, with daily solution changes. Three ex vivo dermises for each treatment were incubated at 4° C., 37° C., or 42° C. The decellularized dermises were fixed overnight in 10% formalin, paraffin embedded and sections at 8 μm. H&E and Elastin van Gieson staining were performed to confirm decellularization and to visualize elastin fibers, respectively. Elastin fiber density quantification was performed via thresholding the elastin fibers using NIH ImageJ software and measurements were recorded.

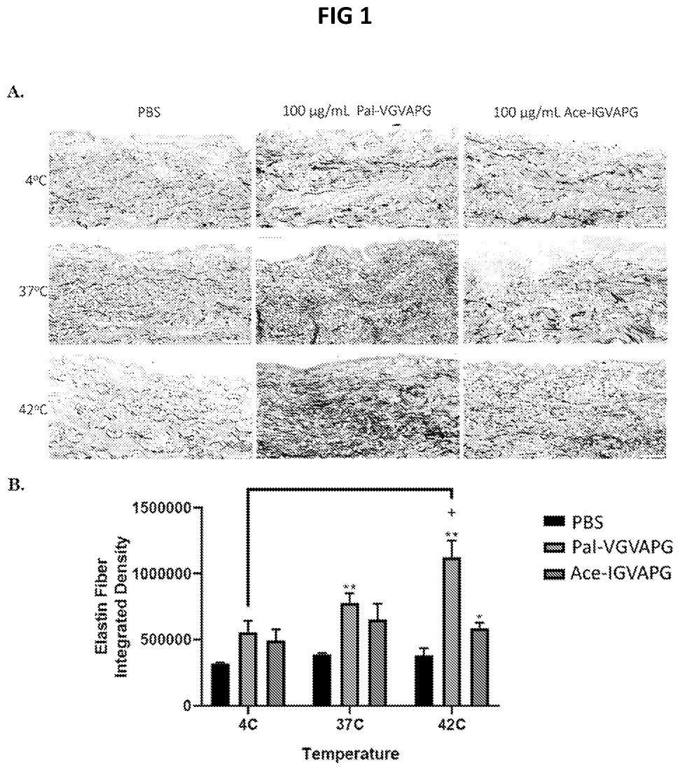

H&E staining confirmed that the tissues were successfully decellularized (data not shown). The absence of cellular nuclei demonstrated that the dermises were incapable of de novo synthesis of elastin. FIG. 1A indicates the representative images of Elastin van Gieson stained decellularized dermises which were incubated with Pal-VGVAPG and Ace-IGVAPG at different temperatures while concentration was kept constant (100 μg/mL). Elastin fibers are stained black at a scale bar of 50 μm.

The Elastin van Gieson staining showed temperature driven co-assembling of the ELPs to existing elastin fibers compared to dermises submerged in PBS as shown in FIG. 1A. The results show a modest increase was observed when the dermises were incubated at 4° C. while higher temperatures of 37° C. and 42° C. showed more pronounced elastin staining. Dermises submerged in Pal-VGVAPG displayed an increase in signal as temperature increased whereas Ace-IGVAPG peptide exhibited the highest increase at 37° C. Dermises submerged in PBS showed similar baseline intensity regardless of temperature as shown in FIG. 1A.

FIG. 1B shows the quantification of elastin fiber density (arbitrary units) in decellularize dermises, where means were considered significant for values p<0.05, **p<0.01 when compared to PBS of the same temperature and +p<0.05 when compared within the same treatment at different temperatures. Statistical significance of differences between means was performed using two-way ANOVA followed by Tukey's multiple comparisons test. Error bars represent standard error of the mean (SEM). All analyses were performed using Prism 8 software (GraphPad, San Diego, CA, U.S.A.).

Results show that concurrent with qualitative histological observations, elastin fiber density analysis of the Elastin van Gieson stain confirmed a significant increase in the integrated density at 37° C. and 42° C. when the dermises were treated with Pal-VGVAPG, while treatment with Ace-IGVAPG showed a significant increase at 42° C. compared to the PBS treated samples of the same temperature. Moreover, a significant difference was observed between dermises treated with Pal-VGVAPG at 42° C. vs 4° C.

The results of FIGS. 1A and 1B indicate that ELPs co-assemble to existing elastin fibers and the assembly is positively regulated with increasing temperature. These results also suggest that the increase in elastin staining in decellularized dermises is due to self-assembly and temperature acts as a catalyst or trigger for assembly within the skin layers.

### Example 4: Concentration Driven Self-Assembly within the Skin

Self-assembly of biomolecules is dependent on a critical self-assembly concentration, where self-assembly biomolecules remain as monomers below a critical concentration and assemble above a specific concentration threshold. Leveraging in vitro critical concentration, we show that increasing concentration of ELPs increases the elastin fiber density suggestive of enhanced non-covalent interactions of ELPs with pre-existing elastin fibers.

To demonstrate concentration driven self-assembly, abdominal skin explants were procured and stabilized overnight, as stated in example 3, and biopsied. Biopsies (12-mm) were treated topically with various concentrations of Ace-IGVAPG or Pal-VGVAPG (12.5, 25, 50 μg/mL) while being maintained in DMEM containing 10% FBS and 1% penicillin/streptomycin/amphotericin B. The skins were formalin fixed, paraffin embedded, sectioned at 8 μm and stain for elastin with the Elastin van Gieson stain.

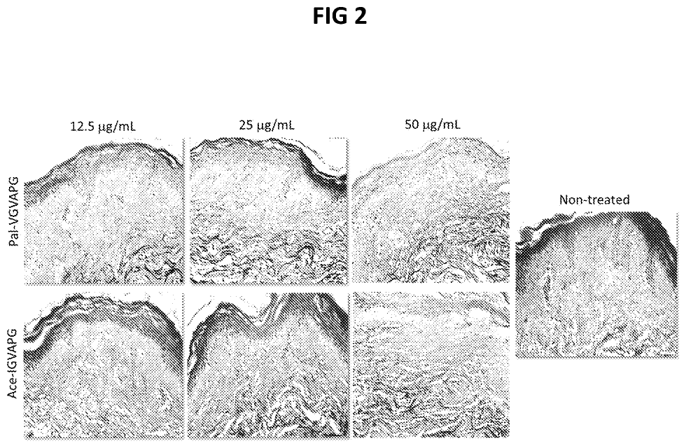

FIG. 2 shows the representative images of ex vivo skin treated topically with various concentrations of the ELPs, Pal-VGVAPG and Ace-IGVAPG. The Elastin van Gieson stain shows an intensification of elastin (dark black fibers) with increasing concentration at a scale bar of 20 μm.

The results of FIG. 2 indicate that histological evaluation of skins treated with the ELPs showed a direct correlation between elastin fiber staining intensity and concentration of Pal-VGVAPG and Ace-IGVAPG, where elastin intensity increased at 12.5 μg/mL, 25 μg/mL and 50 μg/mL, respectively of the peptide amphiphile, Pal-VGVAPG. Similarly, the results showed an increase in elastin intensity at 25 μg/mL and at 50 μg/mL of Ace-IGVAPG, respectively.

### Example 5: pH and Salinity Dependent Peptide Self-Assembly within Intact Ex Vivo Skin

Both exogenous and endogenous factors control self-assembly, two of them being pH and salinity. Skin has a pH gradient, where the surface of the skin is approximately between pH 4.2-5.6, there is a notable increase to pH 6.8 at the interface of the stratum corneum and stratum granulousm and the pH continues to increase to 7.4 in the dermal layer. Skin also has differences in salt content, where interstitial NaCl levels are approximately 0.9% and that of the sweat secretions on the surface of the skin are approximately 0.2%, however, surface salt content is dependent on acclimatization.

To aid in visualization of the effects of pH and salinity on peptide self-assembly within tissue, peptides were labeled via F-moc chemistry with propargylglycine. Biopsies (12-mm) of abdominal skin explants were isolated, maintained, as stated in example 4, and were treated topically with the propargylglycine labeled peptides (8 IAL), twice a day for 5 days. The propargylglycine labeled peptides included A6K, IEIK-13, KLD-12, RADA-16, IGVPAG, and VGVPAG.

To determine how pH and salinity influences peptide assembly in the skin, the propargylglycine labeled peptides were prepared at various pH or salinity pH 3 to 9 or containing a salt content of 0.1-3.0%, respectively. The skins were fixed, cryoprotected, cryosectioned (8 μm) and stored at −20° C. until Copper(I)-Catalyzed Alkyne-Azide Cycloaddition (CuAAC), or click chemistry, was performed to detect peptide assembly. Representative results are shown herein.

Click chemistry was performed by incubating the tissue sections for 1 hour with a working solution of Alexa Fluor-594 Azide (12.5 μM), Sodium Ascorbate (2.5 mM), Copper(II) Sulfate Pentahydrate (75 μM) and Tris (3-hydroxypropyltriazolymethyl) amine (THPTA, 150 μM). The sections were washed, mounted with Fluoromount-G plus DAPI, and were imaged using the Zeiss Axio Observer Z1 Fluorescence Motorized Microscope.

Click chemistry is a technique that uses two biologically inert functional groups, an alkyne and an azide, which form a triazole in the presence of Copper(I). The alkyne on the terminus of the peptide negligibly impacts the aspects of molecule that will impact the penetration of the peptide in tissue (i.e. molecular weight, log P, isoelectric point, and self-assembly) allowing for the peptide to penetrate based on its inherent properties. Once the alkyne-peptide is delivered to the tissue, the sample is fixed, and “stained” with an azido-fluorophore. The tissue is incubated with the azido-fluorophore, Copper(II) Sulfate Pentahydrate (the catalyst), THPTA (the ligand), Sodium Ascorbate (the reducing agent), together this system forms an active Copper(I) complex that is able to form a triazole between the alkyne of the peptide and the azide of the fluorophore. Because CuAAC is a fast, chemoselective, bioorthogonal reaction, only the peptides tagged with the alkyne are reactive leaving the tissue unaffected and intact. This click histology protocol allows for the peptide of interest to be selectively detected via fluorescence microscopy in the presence of endogenous peptides and/or proteins.

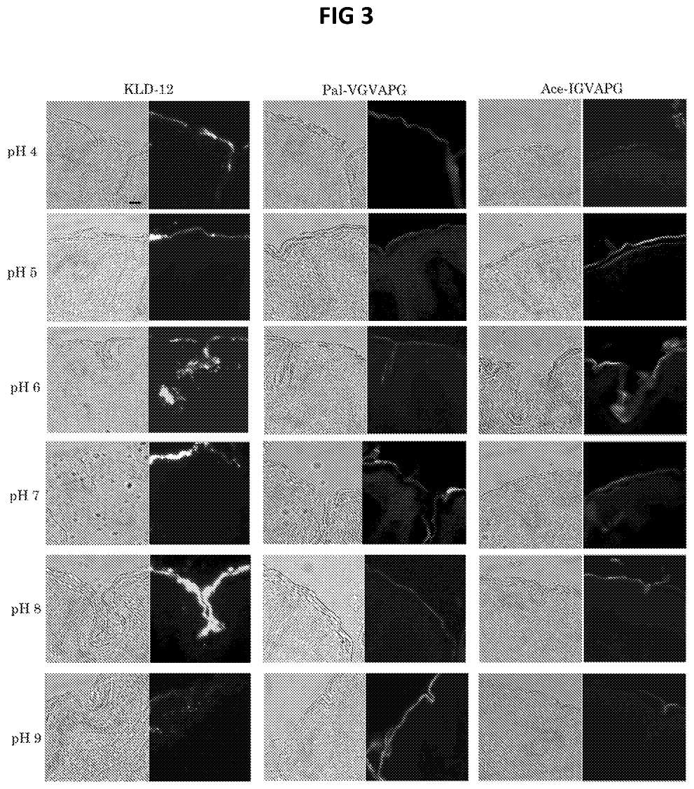

Micrographs of skin treated topically with propargylglycine versions of KLD-12, Pal-VGVAPG and Ace-IGVAPG at pH 4 to 9 at constant concentration (0.6 mg/mL). Brightfield (left panels) and Alexa Fluor 594 clicked peptides (right panels) are shown in FIG. 3 at a scale bar of μm.

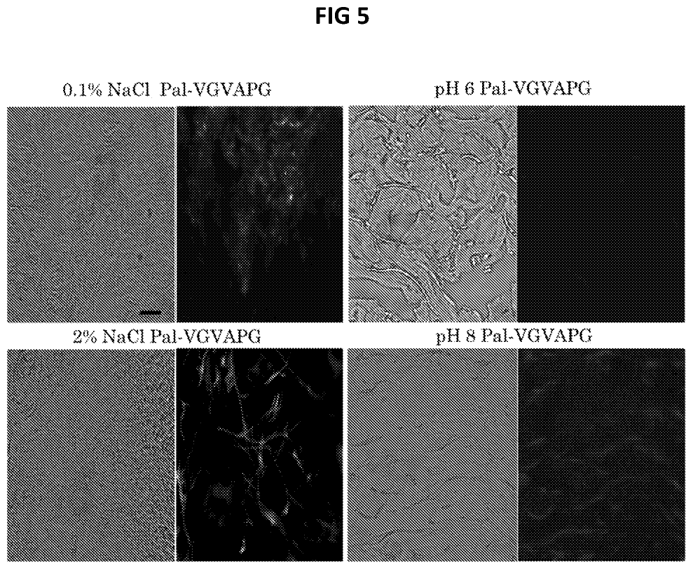

FIG. 3 shows the results of click histology, which revealed that at all pH levels the peptides intercalated and assembled within the stratum corneum. However, at specific pH levels peptide self-assembly was also observed within the lower layers of the skin, as seen with KLD-12 at pH 6 shown in FIG. 3, Pal-VGVAPG at pH 6 and 8 shown in FIG. 5 and Ace-IGVAPG at pH 5 and 7 (data not shown).

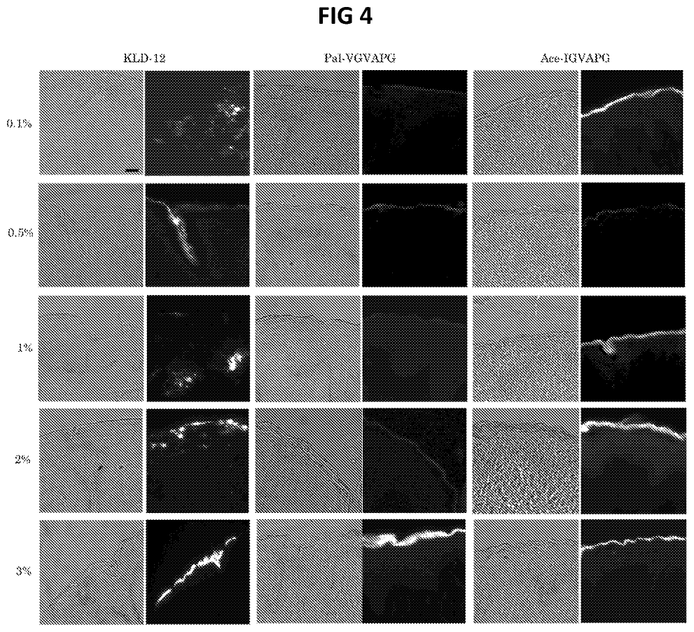

FIG. 4 shows the micrographs of ex vivo skin treated topically with propargylglycine versions of KLD-12, Pal-VGVAPG and Ace-IGVAPG (0.6 mg/mL) at differing ionic strength via altering salt content (0.1%, 0.5%, 1, 2%, 3% NaCl). Brightfield (left panels) and Alexa Fluor 594 clicked peptides (right panels) are shown at a scale bar of 20 μm.

The results of FIG. 4 show that when the effects of salinity were assessed on peptide assembly within the skin, the peptides assembled within the stratum corneum at differing NaCl concentrations. At specific salt concentrations peptide assembly was seen within underlying skin layers as seen with KLD-12 at levels of 0.1, 1 and 2% NaCl shown in FIG. 4 and with Pal-VGVAPG at levels of 0.1 and 2% NaCl shown in FIG. 5.

The combined representative results of Pal-VGVAPG for pH and salinity associated with dermal staining are shown in FIG. 5. As described above, the click histology results revealed that at specific pH and salinity levels peptide self-assembly was also observed within the lower layers of the skin, i.e., dermal layer.

### Example 6: Increased Skin Elasticity Associated with ELP Self-Assembly in Ex Vivo Skin

To demonstrate a cosmetic benefit of increased skin elasticity via self-assembly and co-assembly of peptides, ex vivo skin biopsies (12-mm) were treated topically with Ace-IGVAPG. Following 24 hours, the TA.XT Plus Texture Analyzer was used to indent ex vivo skin with a 7-mm cylindrical probe with 40% strain and a withdrawal rate of 0.1 mm/s. The TA.XT Plus software generated a curve representative of the force (g) applied to the probe by the biopsy overtime (sec) and the areas under the curve for the duration of indentation and the withdrawal time of the probe were used to calculate the percent recovery.

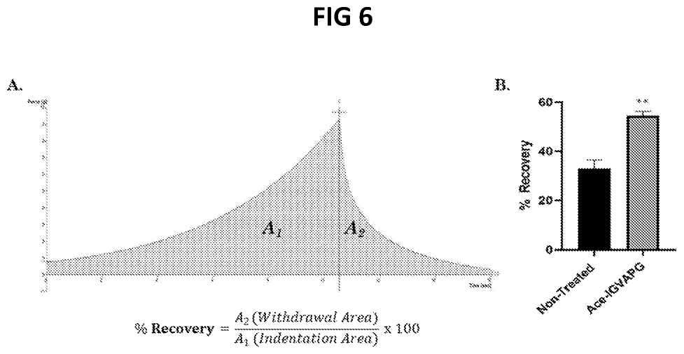

The percent recovery is the ratio of the area under the curve during the withdrawal phase (A₂) to that of the indentation phase (A₁), as seen in an exemplary FIG. 6A.

FIG. 6B shows the results of the cosmetic benefit of enhanced skin elasticity. A purely elastic material has a percent recovery of 100%. Since skin is viscoelastic, its recovery will never be equal to a pure elastic material, ex vivo skin, for example, has a percent recovery of 25% to 35%. As an example, the FIG. 6A shows an exemplary curve generated by the Exponent Connect software associated with the texture analyzer and the percent recovery equation that shown below the curve.

The FIG. 6B shows the percent recovery of ex vivo skin biopsies after 24 h of topical treatment with Ace-IGVAPG (25 mg/mL). Error bars represent SEM (standard error of the mean), where **p<0.01 by two-tailed t test. All analyses were performed using Prism 8 software (GraphPad, San Diego, CA, U.S.A.). The results suggested that the skins treated with 25 μg/mL of Ace-IGVAPG have a significantly higher percent recovery (p=0.0056) when compared to non-treated controls indicative of increased elasticity of ex vivo skin biopsies. Thus, the results of FIG. 6B show that the topical treatment with Ace-IGVAPG (25 μg/mL) provided about 25% greater recovery than non-treated samples after 24 h topical treatment.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0.001 to 30%, or where the pH of an aqueous solution can range from 3 to 9, those inherent limits are specifically disclosed. Where a value is explicitly recited, it is to be understood that values which are about the same quantity or amount as the recited value are also within the scope of the invention, as are ranges based thereon. Where a combination is disclosed, each sub-combination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of re-structuring the skin without cellular biogenesis by affecting macrostructures within the skin, the method comprising effectuating self-assembly within the skin layers by applying a cosmetic composition comprising at least one self-assembly material, wherein the self-assembly material forms functional macrostructures within the skin;
wherein the self-assembly material is a peptide having a chemical structure of R¹-KTTKS-R² (SEQ.ID.NO.: 3) or Palmitoyl Pentapeptide-4;
wherein R¹ is selected from the group consisting of 5-carboxyfluoroscein, 5-carboxyfluoroscein aminohexanoic acid, aminobenzoic acid, acetyl, acryl. benzoyl, biotin, biotin aminohexanoic acid, tert-butyloxycarbonyl, bromoacetyl, bovine serum albumin, carboxybenzyl, 5-(dimethylamino)naphthalene-1-sulfonyl aminohexanoic acid, decanoic acid, diethylenetriaminepentaacetic acid, fatty acids, fluorescein isothiocyanate, fluorescein isothiocyanate aminohexanoic acid, fluorenylmethyloxycarbonyl, formylation, hexanoic acid, hydrazinonicotinic acid, keyhole limpet hemocyanin, lauric acid, lipoic acid, maleimide, 7-methoxycoumarin-4-acetic acid, myristoyl, octanoic acid, palmitoyl, polyethylene glycol, stearic acid, and succinylation;
wherein R 2 is selected from the group consisting of 7-amino-4-(trifluormethyl)-2-benzopyrone, 7-amino-4-methylcoumarinyl, amidation, bovine serum albumin, benzyl, cysteamide, ester, keyhole limpet hemocyanin, multiple antigenetic peptides, methyl, ethylamide, isoamyl amido, N-methyl, hydroxysuccinimide ester, ovalbumin, p-Nitroanilide, and tert-Butyl;
wherein the self-assembling material forms functional macrostructures within the skin following a trigger;
wherein the trigger comprises the pH value about 5, temperature about 37° C., and concentration of >0.5% w/w; and
wherein the self-assembling material is delivered within the skin layers, assembles into fibers of elastin upon the trigger.

2. The method of claim 1, further comprising effectuating the self-assembly of the at least one self-assembly material in vivo within the skin while substantially preventing the self-assembly of the at least one self-assembly material present in the cosmetic composition prior to the application.

3. The method of claim 2, wherein the self-assembly material comprises at least one self-assembling oligomer.

4. The method of claim 2, wherein the self-assembly material comprises more than one oligomer capable of self-assembling with each other within the skin.

5. The method of claim 2, wherein the method provides at least one cosmetic skincare, hair care or make up benefit.

6. The method of claim 5, wherein the cosmetic benefit comprises improving wrinkles, fine lines, pores, sagginess, anti-aging, bulking/plumping, filling, barrier strengthening, smoothing, firming, lifting, radiance, optical properties or glow.

7. The method of claim 1, wherein the functional macrostructures are three dimensional.

8. The method of claim 1, wherein the trigger further comprises an application of a second composition.

9. The method of claim 1, wherein the trigger further comprises a change in pH in a range of about 0.001 to 5 pH units.

10. The method of claim 1, wherein the trigger further comprises a temperature gradient, pH gradient, salt gradient, or concentration gradient.

11. The method of claim 1, wherein the self-assembly is effectuated after the cosmetic composition is applied topically.

12. The method of claim 11, wherein the self-assembly is effectuated within 6 hours following the topical application.

13. The method of claim 11, wherein the self-assembly is effectuated within 4 hours following the topical application.

14. The method of claim 8, wherein the second composition is applied prior to the application of the cosmetic composition.

15. The method of claim 8, wherein the second composition is applied following the application of the cosmetic composition.
